SEQUENCE LISTING (17 sequences)

SEQ ID NO: 1 - protein, 40 aa, synthetic construct
  VARIANT 5: note = T or I
  VARIANT 8: note = Y, V, L, or a lysine residue substituted with a modifying group, or a C-terminal amino acid or an amino acid ester or amino acid amide thereof
  VARIANT 10: note = I or S
  VARIANT 11: note = Y, 2-amino-3-(4-hydroxyphenyl)-2-methylpropanoic acid, Q, A, or 2-aminoisobutyric acid
  VARIANT 12: note = L, M, or 2-amino-2-methylpentanoic acid
  VARIANT 13: note = D or E
  VARIANT 14: note = K, G, R, or E
  VARIANT 15: note = Q or I
  VARIANT 16: note = A, H, or R
  VARIANT 17: note = A, Q, or V
  VARIANT 18: note = A, 2-aminoisobutyric acid, a lysine residue substituted with a modifying group, or a C-terminal amino acid or an amino acid ester or amino acid amide thereof, K, or Q
  VARIANT 19: note = A, D, E, 2-aminoisobutyric acid, or L
  VARIANT 20: note = F or A
  VARIANT 21: note = V or I
  VARIANT 22: note = N, A, Q, a lysine residue substituted with a modifying group, or a C-terminal amino acid or an amino acid ester or amino acid amide thereof, or E
  VARIANT 25: note = I, L or V
  VARIANT 26: note = A, K, or I
  VARIANT 27..30: note = QR, GRG-lysine residue substituted with a modifying group, or a C-terminal amino acid or an amino acid ester or amino acid amide thereof, Q, or G
  VARIANT 40: note = S or absent
EGTFXSDXSXXXXXXXXXXXXXWLXXXXXXGPSSGAPPPX

SEQ ID NO: 2 - protein, 37 aa, synthetic construct
  VARIANT 5: note = T or I
  VARIANT 8: note = Y, V, or L
  VARIANT 10: note = I or S
  VARIANT 11: note = Y, Q, or A
  VARIANT 12: note = L, M, or 2-amino-2-methylpentanoic acid
  VARIANT 13: note = D or E
  VARIANT 14: note = K, G, or E
  VARIANT 17: note = A or V
  VARIANT 18: note = 2-aminoisobutyric acid or K
  VARIANT 19: note = E or L
  VARIANT 21: note = V or I
  VARIANT 22: note = N, A, or E
  VARIANT 25: note = L or V
  VARIANT 26: note = A or K
EGTFXSDXSXXXXXQAXXXFXXWLXXGGPSSGAPPPS

SEQ ID NO: 3 - protein, 37 aa, synthetic construct; identical to SEQ ID NO: 2
  VARIANT 5: note = T or I
  VARIANT 8: note = Y, V, or L
  VARIANT 10: note = I or S
  VARIANT 11: note = Y, Q, or A
  VARIANT 12: note = L, M, or 2-amino-2-methylpentanoic acid
  VARIANT 13: note = D or E
  VARIANT 14: note = K, G, or E
  VARIANT 17: note = A or V
  VARIANT 18: note = 2-aminoisobutyric acid or K
  VARIANT 19: note = E or L
  VARIANT 21: note = V or I
  VARIANT 22: note = N, A, or E
  VARIANT 25: note = L or V
  VARIANT 26: note = A or K

SEQ ID NO: 4 - protein, 37 aa, synthetic construct
  SITE 18: note = 2-aminoisobutyric acid
EGTFTSDYSIYLDKQAAXEFVNWLLAGGPSSGAPPPS

SEQ ID NO: 5 - protein, 38 aa, synthetic construct
  SITE 18: note = 2-aminoisobutyric acid
  SITE 38: note = N6-[N-(1-oxohexadecyl)-L-gamma-glutamyl]-L-lysinami de
EGTFTSDYSIYLDKQAAXEFVNWLLAGGPSSGAPPPSK

SEQ ID NO: 6 - protein, 37 aa, synthetic construct
  SITE 1: note = (S)-2-yl-succinic acid modified residue
  SITE 17: note = 2-aminoisobutyric acid
  SITE 37: note = N6-[N-(1-oxohexadecyl)-L-gamma-glutamyl]-L-lysinami de
GTFTSDYSIYLDKQAAXEFVNWLLAGGPSSGAPPPSK

SEQ ID NO: 7 - protein, 37 aa, synthetic construct; identical to SEQ ID NO: 6
  SITE 1: note = (R)-2-yl-succinic acid modified residue
  SITE 17: note = 2-aminoisobutyric acid
  SITE 37: note = N6-[N-(1-oxohexadecyl)-L-gamma-glutamyl]-L-lysinami de

SEQ ID NO: 8 - protein, 37 aa, synthetic construct; identical to SEQ ID NO: 6
  SITE 1: note = (S)-2-yl-hexanedioic acid modified residue
  SITE 17: note = 2-aminoisobutyric acid
  SITE 37: note = N6-[N-(1-oxohexadecyl)-L-gamma-glutamyl]-L-lysinami de

SEQ ID NO: 9 - protein, 37 aa, synthetic construct; identical to SEQ ID NO: 6
  SITE 1: note = (S)-2-yl-2-methylpentanedioic acid modified residue
  SITE 17: note = 2-aminoisobutyric acid
  SITE 37: note = N6-[N-(1-oxohexadecyl)-L-gamma-glutamyl]-L-lysinami de

SEQ ID NO: 10 - protein, 37 aa, synthetic construct; identical to SEQ ID NO: 6
  SITE 1: note = (S)-2-yl-4-(1H-tetrazol-5-yl)butanoic acid modified residue
  SITE 17: note = 2-aminoisobutyric acid
  SITE 37: note = N6-[N-(1-oxohexadecyl)-L-gamma-glutamyl]-L-lysinami de

SEQ ID NO: 11 - protein, 37 aa, synthetic construct; identical to SEQ ID NO: 6
  SITE 1: note = (R)-2-yl-4-(1H-tetrazol-5-yl)butanoic acid modified residue
  SITE 17: note = 2-aminoisobutyric acid
  SITE 37: note = N6-[N-(1-oxohexadecyl)-L-gamma-glutamyl]-L-lysinami de

SEQ ID NO: 12 - protein, 37 aa, synthetic construct; identical to SEQ ID NO: 6
  SITE 1: note = (S)-2-yl-3-(1H-tetrazol-5-yl)propanoic acid modified residue
  SITE 17: note = 2-aminoisobutyric acid
  SITE 37: note = N6-[N-(1-oxohexadecyl)-L-gamma-glutamyl]-L-lysinami de

SEQ ID NO: 13 - protein, 37 aa, synthetic construct; identical to SEQ ID NO: 6
  SITE 1: note = (R)-2-yl-3-(1H-tetrazol-5-yl)propanoic acid modified residue
  SITE 17: note = 2-aminoisobutyric acid
  SITE 37: note = N6-[N-(1-oxohexadecyl)-L-gamma-glutamyl]-L-lysinami de

SEQ ID NO: 14 - protein, 38 aa, synthetic construct
  SITE 11: note = 2-amino-3-(4-hydroxyphenyl)-2-methylpropanoic acid
  SITE 18: note = 2-aminoisobutyric acid
  SITE 38: note = N6-[N-(1-oxohexadecyl)-L-gamma-glutamyl]-L-lysinami de
EGTFTSDYSIXLDKQAAXEFVNWLLAGGPSSGAPPPSK

SEQ ID NO: 15 - protein, 37 aa, synthetic construct; identical to SEQ ID NO: 6
  SITE 1: note = (S)-2-yl-3-(3-hydroxyisoxazol-5-yl)propanoic acid modified residue
  SITE 17: note = 2-aminoisobutyric acid
  SITE 37: note = N6-[N-(1-oxohexadecyl)-L-gamma-glutamyl]-L-lysinami de

SEQ ID NO: 16 - protein, 38 aa, synthetic construct; identical to SEQ ID NO: 5
  SITE 1: note = 2-((2-oxo-2-((2-(2-oxopiperidin-1-yl)ethyl)amino)et hyl)thio)acetic acid modified residue
  SITE 18: note = 2-aminoisobutyric acid
  SITE 38: note = N6-[N-(1-oxohexadecyl)-L-gamma-glutamyl]-L-lysinami de

SEQ ID NO: 17 - protein, 38 aa, synthetic construct; identical to SEQ ID NO: 5
  SITE 1: note = Fluorenylmethoxycarbonyl amide modified residue
  SITE 18: note = 2-aminoisobutyric acid
  SITE 38: note = N6-[N-(1-oxohexadecyl)-L-gamma-glutamyl]-L-lysinami de
  REGION 38: note = Residue attached to a resin

(19) United States
(12) Patent Application Publication
(10) Pub. No.: US 2026/0116927 A1
(43) Pub. Date: Apr. 30, 2026

(54) Synthesis of GLP-1R/GIPR Agonists and Precursors Thereof

(71) Applicant: Carmot Therapeutics Inc., South San Francisco, CA (US)

(72) Inventors: Shyam KRISHNAN, South San Francisco, CA (US); Thomas FERRARI, South San Francisco, CA (US); Xiaofang WANG, South San Francisco, CA (US); Rui GAO, South San Francisco, CA (US); Fengping XIAO, South San Francisco, CA (US); Zhifeng XIE, South San Francisco, CA (US); Yanpeng LIU, South San Francisco, CA (US)

(73) Assignee: Carmot Therapeutics Inc., South San Francisco, CA (US)

(21) Appl. No.: 19/429,930

(22) Filed: Dec. 22, 2025

Related U.S. Application Data
(63) Continuation of application No. PCT/US2024/035155, filed on Jun. 22, 2024.
(60) Provisional application No. 63/509,754, filed on Jun. 22, 2023.

(51) Int. Cl. C07K 14/00; B01J 21/18; B01J 23/44; C07D 211/86; C07K 1/04; C07K 1/10

(52) U.S. Cl. CPC C07K 14/001; B01J 21/18; B01J 23/44; C07D 211/86; C07K 1/04; C07K 1/10

(57) ABSTRACT

The present disclosure provides a novel synthetic method for preparing GLP-1R/GIPR agonists. Also provided is a synthetic method for preparing 2-((2-oxo-2-((2-(2-oxopiperidin-1-yl)ethyl)amino)ethyl)thio)acetic acid.

### CROSS-REFERENCE TO RELATED APPLICATIONS

This application is a continuation of International Application PCT/US2024/035155, filed Jun. 22, 2024, which claims the benefit of U.S. Provisional Application 63/509,754, filed Jun. 22, 2023. The disclosures of those applications are incorporated by reference herein in their entirety.

### SEQUENCE LISTING

The instant application contains a Sequence Listing which has been submitted electronically in XML file format and is hereby incorporated by reference in its entirety. Said XML copy, created on Jun. 19, 2024, is named 124921_WO006_SL.xml and is 49,813 bytes in size.

### BACKGROUND OF THE INVENTION

Incretin hormones are hormones that provide glycemic control in response to food intake. Gastric inhibitory polypeptide (“GIP”) and glucagon-like peptide-I (“GLP-1”) are primary incretin hormones secreted from small intestinal L cells and K cells, respectively, on ingestion of glucose or nutrients to stimulate insulin secretion from pancreatic cells. GIP and GLP-1 undergo degradation by dipeptidyl peptidase-4 (DPP-4), and rapidly lose their biological activities (see, e.g., Sieno et al., J Diab Invest. (2013) 4:108-30).

The actions of GIP and GLP-1 are believed to be mediated by their receptors, the GIP receptor (GIPR) and the GLP-1 receptor (GLP-1R), respectively, which both belong to the G-protein coupled receptor family and are expressed in pancreatic cells, as well as in various tissues and organs. GLP-1 activities include, without limitation, stimulation of insulin synthesis and secretion, inhibition of glucagon secretion, and inhibition of food intake. GIP activities include, without limitation, stimulation of glucose-dependent insulin secretion, an increase in cell mass, stimulation of glucagon secretion, and a decrease in gastric acid secretion.

GLP-1 and GLP-1 analogs, acting as agonists of GLP-1R, have been shown to be effective in glycemic control, e.g., in type-2 diabetes. See, e.g., WO 2016/131893. In addition to their insulinotropic effects, GIP and GLP-1 are believed to be involved in various biological processes in different tissues and organs that express GIPR and GLP-IR. Investigations using mice lacking GIPR and/or GLP-1R, as well as mice lacking DPP-4, showed involvement of GIP and GLP-1 in divergent biological activities. The results of these investigations point to involvement of GIP and GLP-1 in treating and/or preventing diabetes-related microvascular complications (e.g., retinopathy, nephropathy and neuropathy) and macrovascular complications (e.g., coronary artery disease, peripheral artery disease and cerebrovascular disease), as well as diabetes-related comorbidity (e.g., obesity, non-alcoholic fatty liver disease, bone fracture and cognitive dysfunction). See, e.g., Sieno at page 108.

There remains a need for chemical entities that modulate the activity of GLP-1R and/or GIPR and for improved methods of synthesizing such chemical entities.

### SUMMARY OF THE INVENTION

The present disclosure provides a method of synthesizing an N-terminal conjugated peptidyl compound of formula (I):

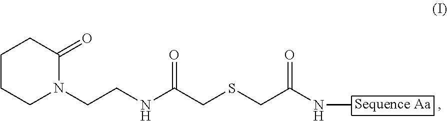 [SMILES: CNC(=O)CSCC(=O)NCCN1CCCCC1=O]

wherein Sequence Aa is a peptide, the method comprising the step of reducing benzyl 2-(2-oxopyridin-1(2H)-yl)ethylcarbamate (XIX):

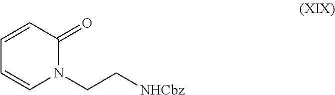 [SMILES: O=C(NCCn1ccccc1=O)OCc1ccccc1]

to 1-(2-aminoethyl)piperidin-2-one (XX):

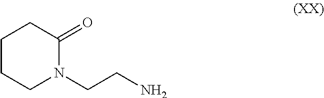

with a catalyst in the presence of H₂ (hydrogen gas). In some embodiments, Sequence Aa comprises the formula W-R⁵, wherein W is a peptide sequence and R⁵ is conjugated to the C-terminus of W, wherein W comprises the following sequence:

(SEQ ID NO: 1)
EGT(Xaa4)(Xaa5)SD(Xaa8)S(Xaa10)(Xaa11)(Xaa12)
(Xaa13)(Xaa14)(Xaa15)(Xaa16)(Xaa17)(Xaa18)
(Xaa19)(Xaa20)(Xaa21)(Xaa22)WL(Xaa25)(Xaa26)
(Xaa27)GPSSGAPPP(Xaa37);

wherein:
Xaa4 is F;
Xaa5 is T or I (e.g., T);
Xaa8 is Y, V, L, or K* (e.g., Y);
Xaa10 is I or S (e.g., I);
Xaa11 is Y, Y*, Q, A, or (Aib) (e.g., Y);
Xaa12 is L, M, or L* (e.g., L);
Xaa13 is D or E (e.g., D);
Xaa14 is K, G, R, or E (e.g., K);
Xaa15 is Q or I (e.g., Q);
Xaa16 is A, H, or R (e.g., A);
Xaa17 is A, Q, or V (e.g., A);
Xaa18 is A, (Aib), K*, K, or Q (e.g., (Aib));
Xaa19 is A, D, E, (Aib), or L (e.g., A, D, E, or L (e.g., E));
Xaa20 is F or A (e.g., F);
Xaa21 is V or I (e.g., V);
Xaa22 is N, A, Q, K*, or E (e.g., N);
Xaa25 is I, L or V (e.g., L);
Xaa26 is A, K, or I (e.g., A);
Xaa27 is Q-R, G-R-G-K*, Q, or G (e.g., G); and
Xaa37 is S or absent (e.g., S); and
   R⁵ is a C-terminal amino acid amide or a C-terminal amino acid that is optionally substituted with 1 or 2 modifying groups selected from an acyl group and a PEG group. W may also comprise the following sequence:

(SEQ ID NO: 4)
EGTFTSDYSIYLDKQAA(Aib)EFVNWLLAGGPSSGAPPPS.

In some embodiments, R⁵ is a C-terminal lysyl amide residue that is optionally substituted with 1 or 2 modifying groups selected from an acyl group and a PEG group. R⁵ may comprise formula (II):

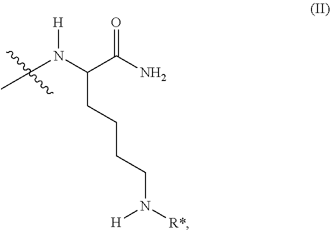

wherein R* comprises the structure (IV):

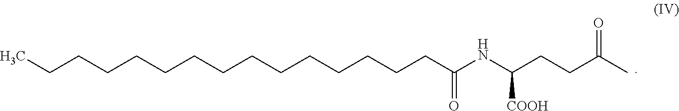 [SMILES: CCCCCCCCCCCCCCCC(=O)N[C@@H](CCC(C)=O)C(=O)O]

In some embodiments, W-R⁵ comprises the structure (V):

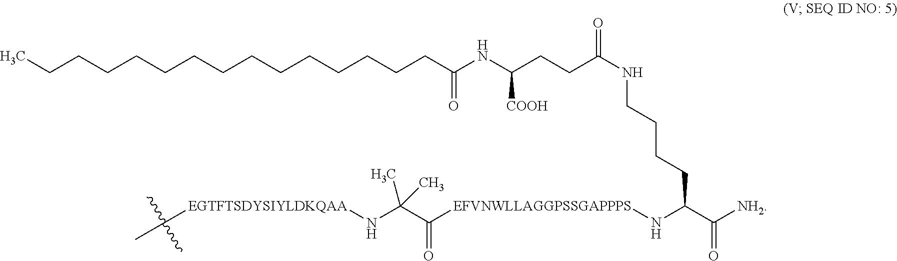

The present method may further comprise the steps of (i) treating the resin-bound peptide of formula (XXIII):

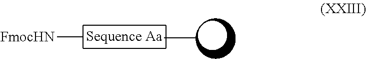

with 20% piperidine in DMF or NMP, optionally wherein the resin-bound peptide is treated with 20% piperidine in DMF or NMP two times, and (ii) reacting the product of step (i) with 2-((2-oxo-2-((2-(2-oxopiperidin-1-yl)ethyl)amino)ethyl)thio)acetic acid (XVII):

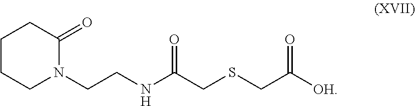 [SMILES: O=C(O)CSCC(=O)NCCN1CCCCC1=O]

under amide bond-forming conditions.

Also provided is a method of synthesizing a compound of formula (XVI):

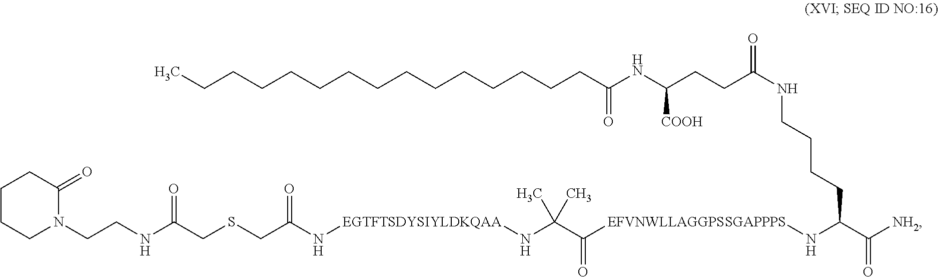

the method comprising the step of reducing benzyl 2-(2-oxopyridin-1(2H)-yl)ethylcarbamate (XIX):

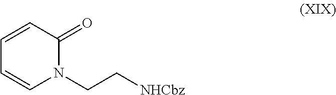 [SMILES: O=C(NCCn1ccccc1=O)OCc1ccccc1]

to 1-(2-aminoethyl)piperidin-2-one (XX):

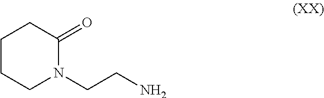

with a catalyst in the presence of H₂ (hydrogen gas). In some embodiments, the method further comprising the steps of (i) treating the resin-bound peptide of formula (XXII):

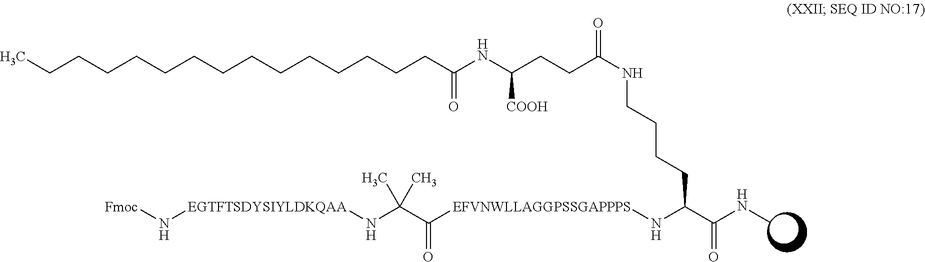

with 20% piperidine in DMF, and (ii) reacting the product of step (i) with 2-((2-oxo-2-((2-(2-oxopiperidin-1-yl)ethyl)amino)ethyl)thio)acetic acid (XVII):

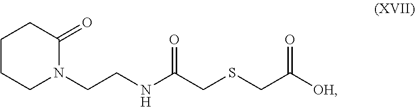 [SMILES: O=C(O)CSCC(=O)NCCN1CCCCC1=O]

under amide bond-forming conditions.

In some embodiments, the amide bond-forming conditions comprise use of a coupling reagent. The coupling reagent may be hexafluorophosphate azabenzotriazole tetramethyl uronium (HATU).

Also provided is a method of synthesizing 2-((2-oxo-2-((2-(2-oxopiperidin-1-yl)ethyl)amino)ethyl)thio)acetic acid (XVII):

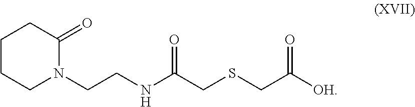 [SMILES: O=C(O)CSCC(=O)NCCN1CCCCC1=O]

the method comprising the step of reducing benzyl 2-(2-oxopyridin-1(2H)-yl)ethylcarbamate (XIX):

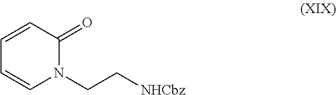 [SMILES: O=C(NCCn1ccccc1=O)OCc1ccccc1]

to 1-(2-aminoethyl)piperidin-2-one (XX):

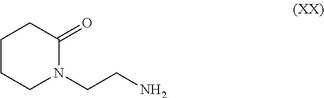

with a catalyst in the presence of H₂ (hydrogen gas).

In some embodiments, the catalyst for reduction is palladium on carbon (Pd/C) or Pd(OH)₂ on carbon (Pd(OH)₂/C).

Also provided is a method of synthesizing an N-terminal conjugated peptidyl compound of formula (I):

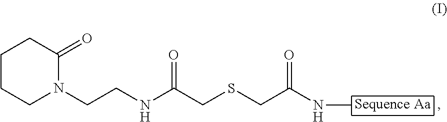 [SMILES: CNC(=O)CSCC(=O)NCCN1CCCCC1=O]

wherein Sequence Aa is a peptide, the method comprising the step of reacting 1-(2-aminoethyl)piperidin-2-one (XX):

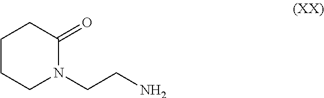

or a salt thereof with 1,4-oxathiane-2,6-dione to produce
2-((2-oxo-2-((2-(2-oxopiperidin-1-yl)ethyl)amino)ethyl)thio)acetic acid (XVII):

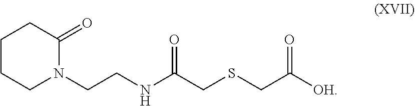 [SMILES: O=C(O)CSCC(=O)NCCN1CCCCC1=O]

In some embodiments, the hydrobromide salt of 1-(2-aminoethyl)piperidin-2-one (XX) is reacted with 1,4-oxathiane-2,6-dione.

The disclosure also provides a method of synthesizing a compound of formula (XVI):

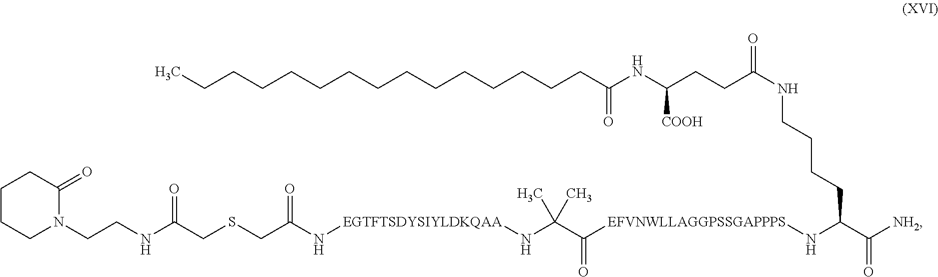

the method comprising the step of reacting 1-(2-aminoethyl)piperidin-2-one (XX):

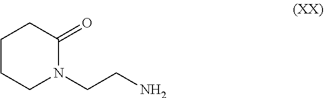

or a salt thereof with 1,4-oxathiane-2,6-dione to produce
2-((2-oxo-2-((2-(2-oxopiperidin-1-yl)ethyl)amino)ethyl)thio)acetic acid (XVII):

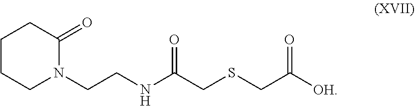 [SMILES: O=C(O)CSCC(=O)NCCN1CCCCC1=O]

In some embodiments, the hydrobromide salt of 1-(2-aminoethyl)piperidin-2-one (XX) is reacted with 1,4-oxathiane-2,6-dione.

Also provided is a method of synthesizing 2-((2-oxo-2-((2-(2-oxopiperidin-1-yl)ethyl)amino)ethyl)thio)acetic acid (XVII):

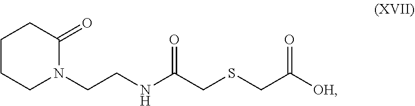 [SMILES: O=C(O)CSCC(=O)NCCN1CCCCC1=O]

the method comprising the step of reacting 1-(2-aminoethyl)piperidin-2-one (XX):

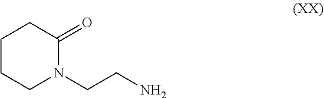

or a salt thereof with 1,4-oxathiane-2,6-dione to produce
2-((2-oxo-2-((2-(2-oxopiperidin-1-yl)ethyl)amino)ethyl)thio)acetic acid (XVII):

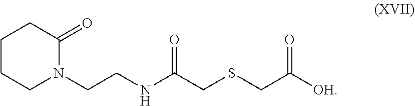 [SMILES: O=C(O)CSCC(=O)NCCN1CCCCC1=O]

In some embodiments, the hydrobromide salt of 1-(2-aminoethyl)piperidin-2-one (XX) is reacted with 1,4-oxathiane-2,6-dione.

In some embodiments, the present method further comprises the step of reacting pyridin-2(1H)-one (XVIII):

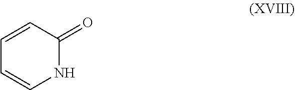

with benzyl (2-bromoethyl)carbamate in the presence of a strong non-nucleophilic base to produce benzyl 2-(2-oxopyridin-1(2H)-yl)ethylcarbamate (XIX):

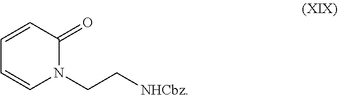 [SMILES: O=C(NCCn1ccccc1=O)OCc1ccccc1]

The strong non-nucleophilic base may be sodium hydride.

In some embodiments, the method further comprises the step of reacting 1-(2-aminoethyl)piperidin-2-one (XX):

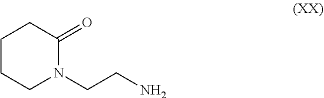

with hydrobromic acid to produce 1-(2-aminoethyl)piperidin-2-one hydrobromide (XXI):

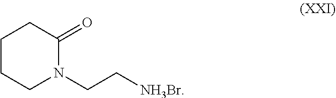

In some embodiments, the method further comprises the step of reacting 1-(2-aminoethyl)piperidin-2-one hydrobromide (XXI):

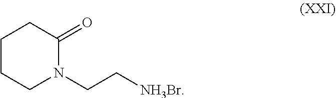

with 1,4-oxathiane-2,6-dione to produce 2-((2-(2-oxopiperidin-1-yl)ethylcarbamoyl) methylthio)acetic acid (XVII):

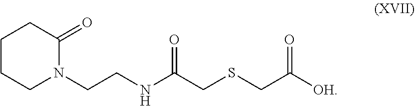 [SMILES: O=C(O)CSCC(=O)NCCN1CCCCC1=O]

Also provided is a compound which is benzyl 2-(2-oxopyridin-1(2H)-yl)ethylcarbamate (XIX):

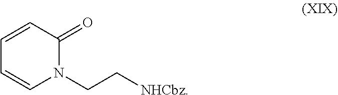 [SMILES: O=C(NCCn1ccccc1=O)OCc1ccccc1]

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### DETAILED DESCRIPTION OF THE INVENTION

The present disclosure provides a novel synthetic method for preparing the N-terminal chemical moiety of a GLP-1R/GIPR agonistic peptide mimetic. This peptide mimetic is represented by formula (I):

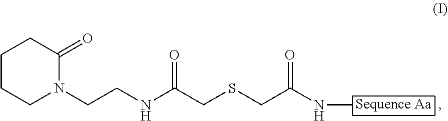 [SMILES: CNC(=O)CSCC(=O)NCCN1CCCCC1=O]

wherein Sequence Aa represents a peptidyl structure. The present synthetic method allows synthesis of the chemical moiety conjugated to Sequence Aa with improved handleability, purity, scalability, reproducibility, and yield.

The agonist herein agonizes the activity of GLP-1 and GIP. As used herein the term “native GLP-1” refers to a peptide comprising the sequence of human GLP-1 (7-36 or 7-37), and the term “native GIP” refers to a peptide comprising the sequence of human GIP (1-42). As used herein, a general reference to “GLP-1” or “GIP” in the absence of any further designation is intended to mean native GLP-1 or native GIP, respectively. In some embodiments, the agonist herein has at least 50% (e.g., at least 60, 70, 80, 90, 95, or 99%) of GLP-1R activation activity of native GLP-1-OH or GLP-1-NH₂ and/or at least 50% (e.g., at least 60, 70, 80, 90, 95, or 99%) of the GIPR activation activity of native GIP.

### I. Sequence Aa

In some embodiments, the peptidyl portion of the compound herein, Sequence Aa, comprises the formula W-R⁵, wherein W represents a peptidyl structure and R⁵ represents a moiety conjugated to the C-terminus of the peptidyl structure.

### A. Peptidyl Structure W

The peptidyl structure W may comprise an amino acid sequence that is present in native GLP-1, with either an —OH or —NH₂ group at the C terminus (i.e., GLP-1-OH or GLP-1-NH₂). The peptidyl structure may also comprise an amino acid sequence present in native GIP. For example, the peptidyl structure may comprise a hybrid sequence having one or more amino acid sequence fragments (e.g., functional fragments) present in native GLP-1 and one or more amino acid sequence fragments (e.g., functional fragments) present in native GIP.

In some embodiments, W has the following formula:

(SEQ ID NO: 1)
EGT(Xaa4)(Xaa5)SD(Xaa8)S(Xaa10)(Xaa11)(Xaa12)
(Xaa13)(Xaa14)(Xaa15)(Xaa16)(Xaa17)(Xaa18)
(Xaa19)(Xaa20)(Xaa21)(Xaa22)WL(Xaa25)(Xaa26)
(Xaa27)GPSSGAPPP(Xaa37),

wherein:
Xaa4 is F;
Xaa5 is T or I (e.g., T);
Xaa8 is Y, V, L, or K* (e.g., Y);
Xaa10 is I or S (e.g., I);
Xaa11 is Y, Y*, Q, A, or (Aib) (e.g., Y);
Xaa12 is L, M, or L* (e.g., L);
Xaa13 is D or E (e.g., D);
Xaa14 is K, G, R, or E (e.g., K);
Xaa15 is Q or I (e.g., Q);
Xaa16 is A, H, or R (e.g., A);
Xaa17 is A, Q, or V (e.g., A);
Xaa18 is A, (Aib), K*, K, or Q (e.g., (Aib));
Xaa19 is A, D, E, (Aib), or L (e.g., A, D, E, or L (e.g., E));
Xaa20 is F or A (e.g., F);
Xaa21 is V or I (e.g., V);
Xaa22 is N, A, Q, K*, or E (e.g., N);
Xaa25 is I, L or V (e.g., L);
Xaa26 is A, K, or I (e.g., A);
Xaa27 is Q-R, G-R-G-K*, Q, or G (e.g., G); and
Xaa37 is S or absent (e.g., S).
   In such embodiments, the nitrogen atom directly connected to Sequence Aa in formula (I) is the amino group of the first glutamate (E) amino acid of W.

In some embodiments, W has the following formula:

(SEQ ID NO: 2)
EGTF(Xaa5)SD(Xaa8)S(Xaa10)(Xaa11)(Xaa12)
(Xaa13)(Xaa14)QA(Xaa17)(Xaa18)(Xaa19)
F(Xaa21)(Xaa22)WL(Xaa25)(Xaa26)GGPSSGAPPPS,

wherein:
Xaa5 is T or I (e.g., T);
Xaa8 is Y, V, or L (e.g., Y);
Xaa10 is I or S (e.g., I);
Xaa11 is Y, Q, or A (e.g., Y);
Xaa12 is L, M, or L* (e.g., L);
Xaa13 is D or E (e.g., D);
Xaa14 is K, G, or E (e.g., K);
Xaa17 is A or V (e.g., A);
Xaa18 is (Aib) or K (e.g., (Aib));
Xaa19 is E or L (e.g., E);
Xaa21 is V or I (e.g., V);
Xaa22 is N, A, or E (e.g., N);
Xaa25 is L or V (e.g., L); and
Xaa26 is A or K (e.g., A).
   In such embodiments, the nitrogen atom directly connected to Sequence Aa in formula (I) is the amino group of the first glutamate (E) amino acid of W.

In some embodiments, W has the following formula:
EGTF(Xaa5)SD(Xaa8)S(Xaa10)(Xaa11)(Xaa12)(Xaa13)(Xaa14)QA(Xaa17)(Aib)(X aa19)F(Xaa21)(Xaa22)WL(Xaa25)(Xaa26)GGPSSGAPPPS (SEQ ID NO:3), wherein each of the “Xaa” variables is as defined above.
   In such embodiments, the nitrogen atom directly connected to Sequence Aa in formula (I) is the amino group of the first glutamate (E) amino acid of W.

In some embodiments, W has the following formula:

(SEQ ID NO: 4)
EGTFTSDYSIYLDKQAA(Aib)EFVNWLLAGGPSSGAPPPS.

In such embodiments, the nitrogen atom directly connected to Sequence Aa in formula (I) is the amino group of the first glutamate (E) amino acid of W.

As used herein, “(Aib)” refers to 2-aminoisobutyric acid (also known as α-aminoisobutyric acid or α-methylalanine or 2-methylalanine).

As used herein, Y* refers to 2-amino-3-(4-hydroxyphenyl)-2-methylpropanoic acid (e.g., (S)-2-amino-3-(4-hydroxyphenyl)-2-methylpropanoic acid).

As used herein, L* refers to 2-amino-2-methylpentanoic acid (e.g., (S)-2-amino-2-methylpentanoic acid) or a C-terminal amino acid or an amino acid ester or an amino acid amide thereof.

As used herein, K* is a lysine residue substituted with a modifying group, or a C-terminal amino acid or an amino acid ester or amino acid amide thereof.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics or substitutions of residues with similar side chain volume are also within the scope of this disclosure.

Amino acids can be grouped according to similarities in the properties of their side chains (see, e.g., A L. Lehninger, in Biochemistry, 2^{nd} Ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H).

Alternatively, naturally occurring residues can be divided into groups based on common side-chain properties: (1) hydrophobic: norleucine, Met, Ala, Val, Leu, Ile, Phe, Trp; (2) neutral hydrophilic: Cys, Ser, Thr, Asn, Gln, Ala, Tyr, His, Pro, Gly; (3) acidic: Asp, Glu; (4) basic: His, Lys, Arg; (5) residues that influence chain orientation: Gly, Pro; (6) aromatic: Trp, Tyr, Phe, Pro, His, or hydroxyproline. Non-conservative substitutions will entail exchanging a member of one of these classes for another class.

In some embodiments, conservative substitutions for use in the variants described herein are as follows: Ala into Gly or into Ser; Arg into Lys; Asn into Gln or into His; Asp into Glu or into Asn; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr or into Phe; Tyr into Phe or into Trp; and/or Phe into Val, into Tyr, into Ile or into Leu.

In general, conservative substitutions encompass residue exchanges with those of similar physicochemical properties (e.g., substitution of a hydrophobic amino acid residue for another hydrophobic amino acid residue).

In some embodiments, W includes one or more naturally occurring amino acids found, e.g., in polypeptides and/or proteins produced by living organisms, such as Ala (A), Val (V), Leu (L), Ile (I), Pro (P), Phe (F), Trp (W), Met (M), Gly (G), Ser (S), Thr (T), Cys (C), Tyr (Y), Asn (N), Gln (Q), Asp (D), Glu (E), Lys (K), Arg (R), and His (H).

In some embodiments, W includes one or more independently selected modifications that occur in so-called modified peptides. Such modifications include, but are not limited to: (i) the incorporation of lactam-bridge; (ii) head-to-tail cyclization; (iii) one or more alternative or non-naturally occurring (D- or L-) amino acids, such as synthetic non-native amino acids, substituted amino acids, and D-amino acids; (iv) peptide bond replacements; (v) targeting groups; and the like. In some embodiments, the peptide includes one modification in either W or R⁵. In other embodiments, the peptide includes more than one independently selected modification (e.g., 2 independently selected modifications, 3 independently selected modifications, 4 independently selected modifications, 5 independently selected modifications, 6 independently selected modifications, 7 independently selected modifications, 8 independently selected modifications, 9 independently selected modifications, or 10 independently selected modifications) that occur in W and/or R⁵ (e.g., in W only; or in R⁵ only; or in both W and R⁵).

Non-limiting examples of alternative or non-naturally amino acids include, D-amino acids, beta-amino acids, homocysteine, phosphoserine, phosphothreonine, phosphotyrosine, hydroxyproline, gamma-carboxyglutamate, hippuric acid, octahydroindole-2-carboxylic acid, statine, 1,2,3,4-tetrahydroisoquinoline-3-carboxylic acid, penicillamine (3-mercapto-D-valine), ornithine, citruline, alpha-methyl-alanine, para-benzoylphenylalanine, para-ammo phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-Icyclopentanecarboxylic acid, 1-amino-i-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof (each which can independently be D- or L-amino acids).

The peptidyl structure W may comprise one or more non-natural peptide bonds. Non-limiting examples of peptide bond replacements include urea, thiourea, carbamate, sulfonyl urea, trifluoroethylamine, ortho-(aminoalkyl)-phenylacetic acid, paras (aminoalkyl)-phenylacetic acid, meta-(aminoalkyl)-phenylacetic acid, thioamide, tetrazole, boronic ester, olefinic group, and derivatives thereof. Unless otherwise indicated, the peptide bonds herein are naturally occurring peptide bonds.

In some embodiments, W includes only naturally occurring amino acids. In other embodiments, W includes only alternative or non-naturally occurring amino acids. In still other embodiments, W includes one or more naturally occurring amino acids and one or more alternative or non-naturally occurring amino acids. In some of the foregoing embodiments, W includes only L-amino acids; or W includes both D- and L-amino acids; or W includes only D-amino acids. While not wishing to be bound by theory, it is believed that the incorporation of D-amino acids can confer enhanced in vivo or intracellular stability to the compounds described herein.

### B. R⁵ Moiety

In some embodiments, R⁵ is a C-terminal amino acid amide that is optionally substituted with 1 or 2 modifying groups (e.g., 1 or 2 groups selected from an acyl group and a PEG group). In other embodiments, R⁵ is a C-terminal amino acid that is optionally substituted with 1 or 2 modifying groups (e.g., 1 or 2 groups selected from an acyl group and a PEG group).

In some embodiments, R⁵ is a C-terminal lysyl amide residue that is optionally substituted with 1 or 2 modifying groups (e.g., 1 or 2 groups selected from an acyl group and a PEG group). In some embodiments, R⁵ is a C-terminal L-lysyl amide residue that is optionally substituted with 1 or 2 modifying groups (e.g., 1 or 2 groups selected from an acyl group and a PEG group).

In some embodiments, R⁵ has the formula (II) or (III):

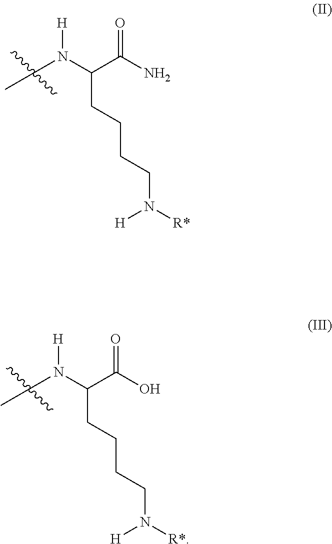

wherein R* is H or a modifying group (e.g., an acyl group or a PEG group). In some embodiments, formula (II) or (III) represents an L-amino acid. In other embodiments, formula (II) or (III) represents a D-amino acid.

In some embodiments, R* is H.

In some embodiments, the modifying group (i.e., R*) is an acyl group. In further embodiments, R* is a C₂₋₃₀ (e.g., C₂₋₂₀, C₂₋₁₀, C₂₋₆) acyl group that is optionally substituted with 1 or 2 independently selected Rf. Each occurrence of Rf may be selected from the group consisting of —C(═O)(OH); —C(═O)(C₂₋₂₀ alkyl); —C(═O)O(C₂₋₂₀ alkyl); —P(═O)(OH)₂; and —S(O)₁₋₂(C₁₋₆ alkyl); oxo; F; C₁₋₁₀ alkoxy; C₁₋₁₀ haloalkoxy; and —N(R^{g})(R^{h}). In some embodiments, each occurrence of Rf is independently selected from the group consisting of —C(═O)(OH) and —N(R^{g})(R^{h}). Each occurrence of R^{g} and R^{h} is independently selected from the group consisting of H; C₁₄ alkyl; —C(═O)(C₂₋₂₀ alkyl); —C(═O)O(C₂₋₂₀ alkyl); and —S(O)₁₋₂(C₁₋₆ alkyl).

In some embodiments, the modifying group (i.e., R*) has the formula (IV):

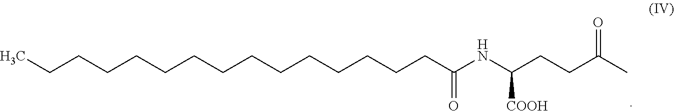 [SMILES: CCCCCCCCCCCCCCCC(=O)N[C@@H](CCC(C)=O)C(=O)O]

### C. Exemplary Sequence Aa

In some embodiments, the W-R⁵ structure herein has the following formula (V):

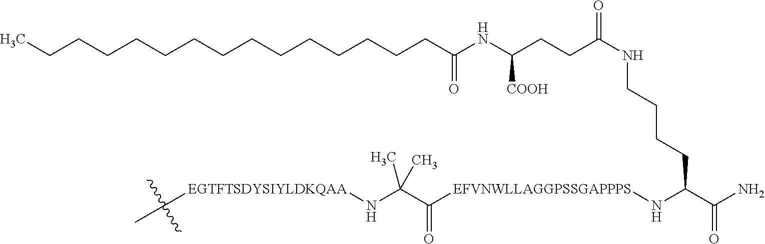

(V; SEQ ID NO:5). In such embodiments, the nitrogen atom directly connected to Sequence Aa in formula (I) is the amino group of the first glutamate (E) amino acid of W.

In some embodiments, the W-R⁵ structure may have any one of the following formulae (VI-XV):

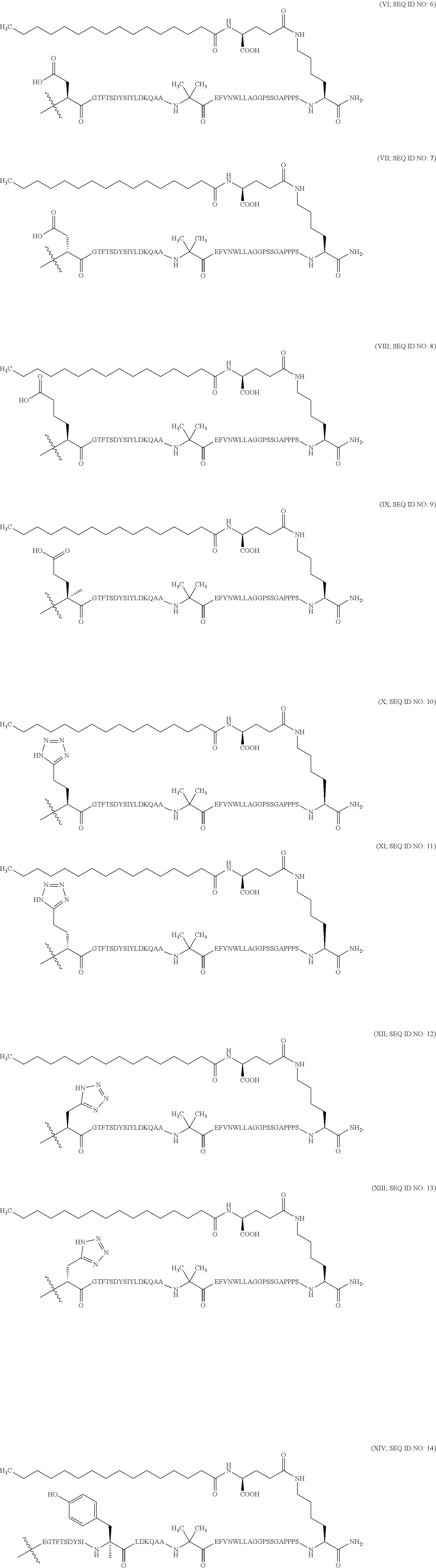

14), wherein the nitrogen atom directly connected to Sequence Aa in formula (I) is the amino group of the first glutamate (E) amino acid of W,
and

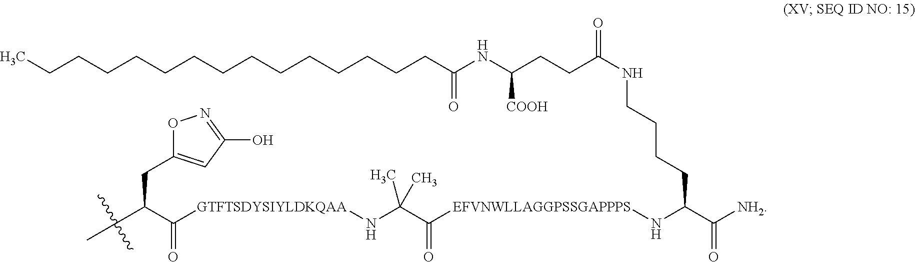

As used in SEQ ID NOs 6-13 and 15, [symbol] indicates the point of attachment of the peptide to the chemical structure in Formula (I).

To illustrate the conjugation between carboxylic acid (XVII) and W-R⁵, a nonlimiting exemplary dual GLP-1R/GIPR agonist has the following structural formula (XVI):

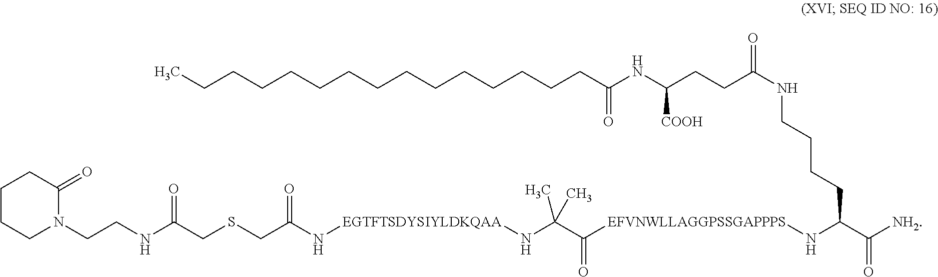

Unless otherwise indicated, the one-letter abbreviations used to describe amino acid residues in SEQ ID NOs: 4-15 are amino acid residues in their native configurations connected by native peptidyl bonds.

### II. Synthesis of N-Terminal Moiety

The dual GLP-1R/GIPR agonist herein may be synthesized by conjugating the N-terminus of Sequence Aa to a compound of formula (XVII) via an amide bond:

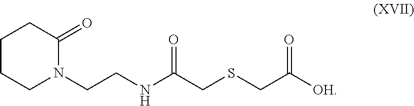 [SMILES: O=C(O)CSCC(=O)NCCN1CCCCC1=O]

This compound, 2-((2-(2-oxopiperidin-1-yl)ethylcarbamoyl)methylthio)acetic acid (formula XVII), is a key intermediate in the synthesis of the agonist compound herein. The present disclosure provides an improved synthetic method for the production of this intermediate using a novel synthetic procedure.

In some embodiments, the present synthesis method comprises one or more steps shown in the following synthetic scheme:

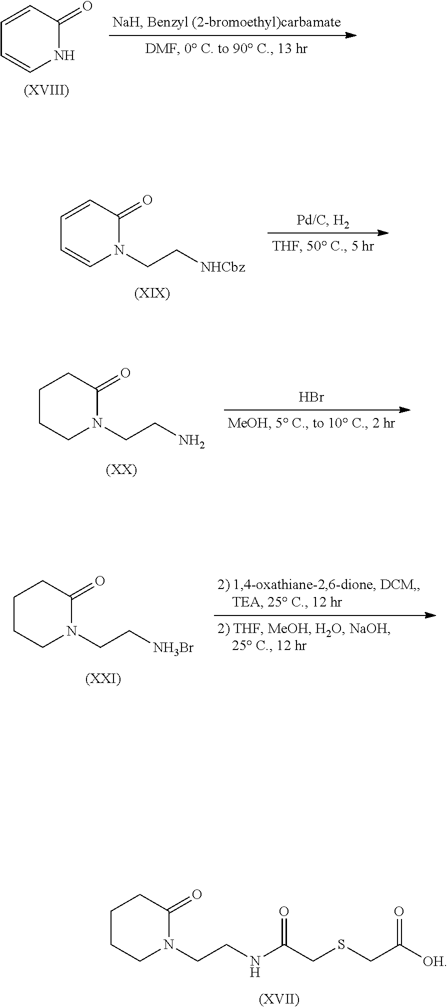

In some embodiments, the present method comprises the step of reacting pyridin-2(1H)-one (XVIII) with benzyl (2-bromoethyl)carbamate in the presence of a strong non-nucleophilic base (e.g., sodium hydride) in a polar aprotic solvent (e.g., dimethylformamide) optionally at a range of temperatures (e.g., 0° C. to 90° C.) to produce benzyl 2-(2-oxopyridin-1(2H)-yl)ethylcarbamate (XIX). In some embodiments, the present method comprises the step of reducing benzyl 2-(2-oxopyridin-1(2H)-yl)ethylcarbamate (XIX) with a catalyst (e.g., palladium on carbon or palladium hydroxide on carbon) under hydrogen gas in a polar aprotic solvent (e.g., THF) or polar protic solvent (e.g., methanol or water or a mixture thereof) optionally at an elevated temperature (e.g., 50° C.) to produce 1-(2-aminoethyl)piperidin-2-one (XX). In some embodiments, the present method comprises the step of producing 1-(2-aminoethyl)piperidin-2-one hydrobromide (XXI) by reacting 1-(2-aminoethyl)piperidin-2-one (XX) with hydrobromic acid in a protic solvent (e.g., methanol) optionally at a reduced temperature (e.g., 5° C. to 10° C.). In some embodiments, the present method comprises reacting 1-(2-aminoethyl)piperidin-2-one hydrobromide (XXI) with 1,4-oxathiane-2,6-dione in a weakly polar solvent or nonpolar solvent (e.g., dichloromethane) in the presence of an organic base (e.g., triethylamine, N,N-diisopropylethylamine, or other trialkyl amine) to produce 2-((2-(2-oxopiperidin-1-yl)ethylcarbamoyl)methylthio)acetic acid (XVII).

In some embodiments, the present method comprises the step of reacting pyridin-2(1H)-one (XVIII) with benzyl (2-bromoethyl)carbamate in the presence of a non-nucleophilic base (e.g., Cs₂CO₃) in a polar aprotic solvent (e.g., MeCN) to produce benzyl 2-(2-oxopyridin-1(2H)-yl)ethylcarbamate (XIX).

In some embodiments, the present method comprises the step of reducing benzyl 2-(2-oxopyridin-1(2H)-yl)ethylcarbamate (XIX) with a catalyst (e.g., 20% Pd(OH)₂ on carbon) under hydrogen gas in a polar protic solvent (e.g., methanol) to produce 1-(2-aminoethyl)piperidin-2-one (XX).

In some embodiments, the present synthesis method comprises the step shown in the following synthetic scheme:

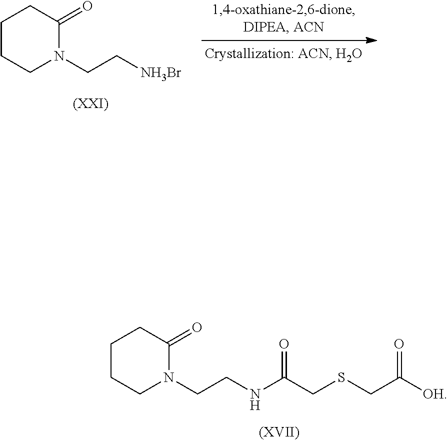

The present treatment methods may comprise the step of reacting 1-(2-aminoethyl)piperidin-2-one (XX):

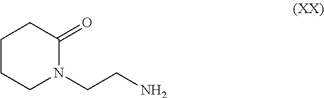

or a salt thereof with 1,4-oxathiane-2,6-dione to produce 2-((2-oxo-2-((2-(2-oxopiperidin-1-yl)ethyl)amino)ethyl)thio)acetic acid (XVII)

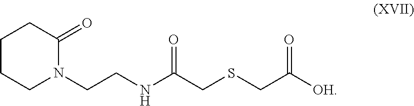 [SMILES: O=C(O)CSCC(=O)NCCN1CCCCC1=O]

In some embodiments, this step is carried out in a polar aprotic solvent (e.g., DCM, MeCN and/or THF). The reaction may be carried out in the presence of a tertiary amine base (e.g., TEA or DIPEA), optionally a non-nucleophilic tertiary amine base (e.g., DIPEA). In some embodiments, the reaction is carried out in MeCN and DIPEA. The product may be recovered in 70% or greater (e.g., 75% or greater or 80% or greater) yield. In some embodiments, about 1.07 molar equivalents (e.g., about 1.01, 1.02, 1.03, 1.04, 1.05, 10.6, 1.08, 1.09, 1.10, 1.11, 1.12, 1.13, or 1.14 molar equivalents) of 1-(2-aminoethyl)piperidin-2-one or a salt thereof (e.g., a hydrobromide salt thereof) are reacted with 1.0 molar equivalents of 1,4-oxathiane-2,6-dione.

In some embodiments, the present synthesis method comprises one or more steps shown in the following synthetic scheme:

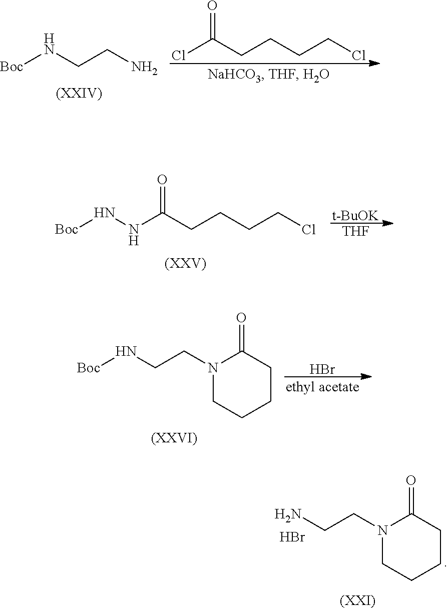

For example, tert-butyl N-(2-aminoethyl)carbamate (XXIV):

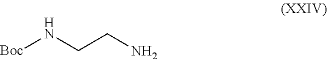 [SMILES: CC(C)(C)OC(=O)NCCN]

may be reacted with 5-chlorovaleryl chloride (i.e., 5-chlorovaleroyl chloride) in the presence of a base (e.g., NaHCO₃) in a polar solvent (e.g., TIHF or H₂O) to produce tert-butyl N-[2-(5-chloropentanoylamino)ethyl]carbamate (XXV):

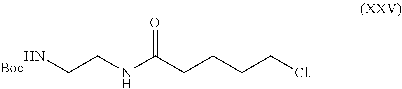 [SMILES: NCCNC(=O)CCCCCl ; "Boc"]

In some embodiments, tert-butyl N-[2-(5-chloropentanoylamino)ethyl]carbamate (XXV) is treated with a base (e.g., tBuOK) in a polar solvent (e.g., THF) to afford tert-butyl 2-(2-oxopiperidin-1-yl)ethylcarbamate (XXVI):

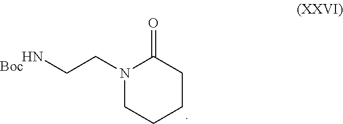

tert-butyl 2-(2-oxopiperidin-1-yl)ethylcarbamate (XXVI) may be reacted with an acid (e.g., HBr) to produce 1-(2-Aminoethyl)piperidin-2-one (XX):

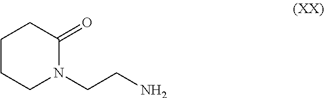

or a salt thereof, such as the hydrobromide salt thereof (XXI):

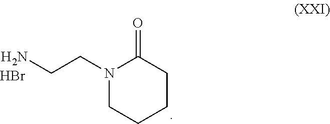

In some embodiments, the present method comprises one or more steps shown in the following synthetic scheme:

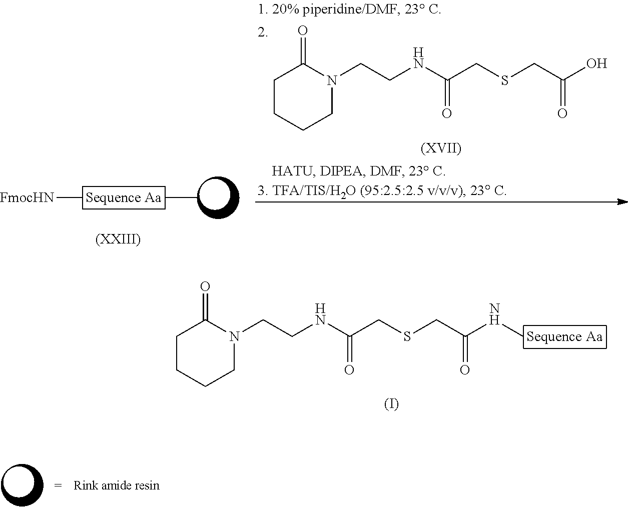

In some embodiments, the present method comprises the step of using standard solid-phase synthetic procedures to produce a resin-bound peptide having the structure (XXII):

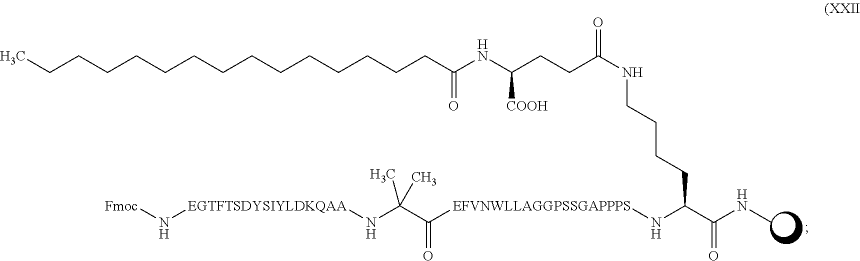

SEQ ID NO: 17), referred to in some embodiments as (XXIII):

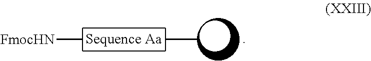

In some embodiments, the present method comprises the step of deprotecting the resin-bound peptide of formula (XXII) or (XXIII) using 20% piperidine in DMF or NMP. In some embodiments, the piperidine-deprotected resin-bound peptide is reacted with intermediate (XVII) under amide bond-forming coupling conditions. The amide bond-forming conditions may involve treating the intermediate (XVII) with a coupling reagent (e.g., hexafluorophosphate azabenzotriazole tetramethyl uronium (HATU)) in the presence of a weak base (e.g., diisopropyl ethyl amine) in a polar aprotic solvent (e.g., dimethyl formamide). In some embodiments, after the intermediate (XVII) has been coupled to the resin-bound deprotected peptide via an amide bond, the conjugated peptide is cleaved from the resin using a strong acid (e.g., trifluoroacetic acid) in the presence of a reducing agent (e.g., triisopropyl silane) in an aqueous solvent.

The carboxylic acid of formula (XVII) may be produced from commercially available starting materials in greater than 5% (e.g., greater than 5%, 6%, 7%, 8%, 9%, 10% 11%, 12%, 13%, 14%, or 15%) yield.

The present methods for synthesizing the peptide (I), GLP-1R/GIPR agonists (VI)-(XVI), and their intermediate (XVII) have several advantages over previously reported synthetic procedures, including enhanced yield and a reduced number of synthetic and/or purification steps to reach the desired compound. In some embodiments, the present methods also avoid the use of hazardous reagents (e.g., hydrazine or diisopropyl azodicarboxylate (DIAD)) or reagents that produce byproducts that are challenging to dispose of (e.g., triphenylphosphine and triphenylphosphine oxide). Furthermore, in some embodiments, the synthetic methods described herein involve the reduction of a pseudoaromatic pyridin-2(1H)-one group with a mild and readily available reducing agent (hydrogen gas in the presence of catalytic Pd/C). The present invention thus streamlines the synthesis of peptide (I), GLP-1R/GIPR agonists (VI)-(XVI), and their intermediate (XVII) compared to previously reported synthetic procedures.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety, as if each individual reference were specifically and individually indicated to be incorporated by reference in its entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In some embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: Synthesis of 2-((2-(2-oxopiperidin-1-yl)ethylcarbamoyl)methylthio)acetic acid (XVII)

The synthesis of 2-((2-(2-oxopiperidin-1-yl)ethylcarbamoyl)methylthio)acetic acid (XVII) was carried out in four steps in 14% overall yield. The overall scheme is shown below.

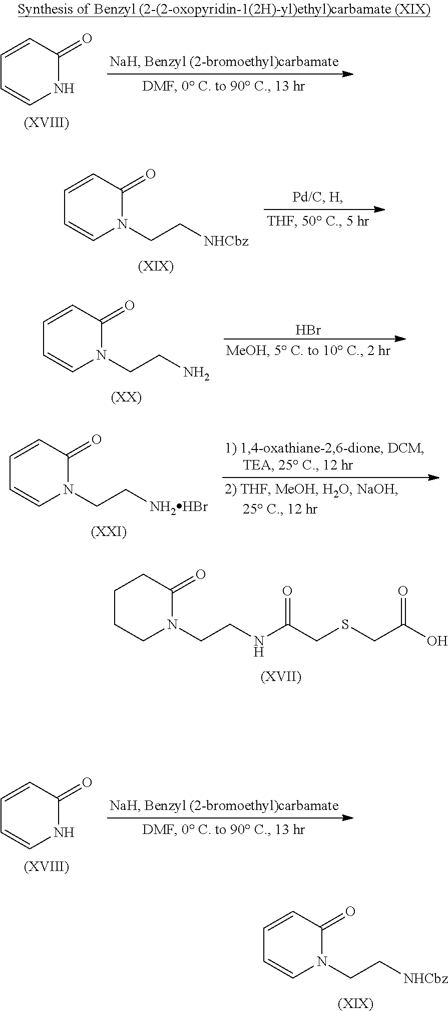

To a solution of pyridin-2(1H)-one (XVIII) (CAS: 142-08-5, 10 g, 105.1 mmol) in anhydrous DMF (100 mL) was added NaH (60% dispersion in mineral oil, 5.1 g, 1.2 eq., 210.3 mmol) in portions at 0° C. under N₂. The mixture was stirred for 0.5 hours. Benzyl (2-bromoethyl)carbamate (CAS: 53844-02-3, 33.92 g, 1.25 eq, 131.43 mmol) was then added in one portion. The reaction mixture was then heated to 90° C. for another 13 hours, at which time the reaction was deemed complete, then quenched with water (50 mL) and extracted with EtOAc (3×100 mL). The combined organic extracts were dried over Na₂SO₄, filtered, and concentrated under reduced pressure to afford a crude residue. The crude residue was purified using reverse phase column chromatography (ACN/H₂O, gradient: 25%-30%) to obtain 7 g of a yellow solid (yield 27.9%). MS (ESI, pos. ion) m/z: 273.1 (M+1).

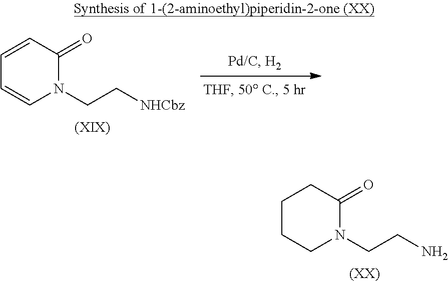

To a solution of benzyl (2-(2-oxopyridin-1(2H)-yl)ethyl)carbamate (XIX) (15 g, 55.08 mmol) in THF (80 mL) was added Pd/C (3 g, 10 wt %). The reaction mixture was heated to 50° C. under H₂ (50 psi) for 5 hours and then filtered through Celite®. The Celite® bed was washed with EtOAc (2×100 mL). The filtrate and washes were combined and concentrated under reduced pressure to afford a pale oil (12 g, 153% yield, purity=50%; impurity was solvent), which was used directly in the next step. MS (ESI, pos. ion) m/z: 142.2 (M+1).

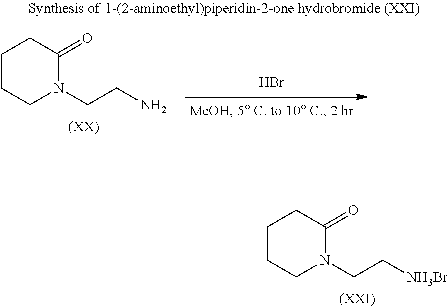

To a solution of 1-(2-aminoethyl)piperidin-2-one (XX) (100 g, 704 mmol, 1.0 equiv) in 500 mL of MeOH that had been pre-cooled using an ice bath was added 40% aqueous HBr (169 g, 845 mmol, 1.2 equiv). The reaction mixture was stirred at 5° C. for 2 hours. The solvent was then removed under reduced pressure and EtOH (1 L) was added. The reaction mixture was concentrated again to remove water. The reside afforded was placed in an ice bath and slowly dissolved in MeOH (300 mL) and MTBE (600 ml). The solution was stirred at 5-10° C. for 2 hours. The reaction mixture was filtered and washed with MTBE (1 L) to afford 1-(2-aminoethyl)piperidin-2-one hydrobromide (XXI) (100 g, purity 98%, yield 64%) as a white solid. MS (ESI, pos. ion) m/z: 143.2 (M+1). ¹H NMR (400 MHz, D₂O) δ 3.61 (t, J=5.9 Hz, 2H), 3.35 (t, J=5.7 Hz, 2H), 3.17 (t, J=5.9 Hz, 2H), 2.33 (t, J=6.3 Hz, 2H), 1.80-1.68 (m, 4H).

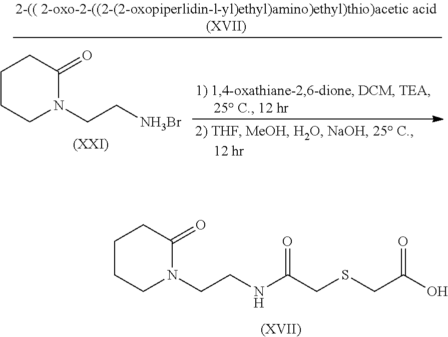

1-(2-aminoethyl)piperidin-2-one hydrobromide (XXI) (90 g, 1 eq) was charged to a 500 mL glass flask with DCM (200 mL). After cooling to 0° C., TEA (85.72 g, 2.1 eq) was added. The reaction mixture was stirred at 0° C. for 0.5 hours. A solution of 1,4-oxathiane-2,6-dione (CAS: 3261-87-8, 106.61 g, 2 eq) in THF (50 mL) was then added dropwise to the mixture. The reaction mixture was stirred at 25° C. for 12 hours under N₂ atmosphere. Ion pair chromatography (IPC):HPLC showed full consumption of 1-(2-aminoethyl)piperidin-2-one. The reaction mixture was then concentrated under reduced pressure and the residue afforded was triturated with EtOAc (7.5 V) for 1 hour and the slurry was filtered. The filter cake was then triturated with CH₃CN (5 V) for 1 hour and filtered. The filter cake was dried in vacuo to give a crude product (260 g). ¹H-NMR of the crude product showed the absence of the excess 1,4-oxathiane-2,6-dione. The 260 g crude product was then dissolved in water (2 V). Two equivalents of sat. NaHCO₃ solution were added and the reaction mixture was stirred for 0.5 hours before addition of another 1 eq. of solid Na₂CO₃. The solution was then concentrated to remove TEA (until absent by ¹H NMR analysis). The residue was dissolved in water (2 V) and the pH of the resulting solution was adjusted to pH 3 with 6 N HCl. The reaction mixture was concentrated to give a residue. The residue was then triturated with DCM/MeOH (10/1 v/v, 5 V) for 30 min. The slurry was then filtered and the filter cake was washed with DCM/MeOH (10/1 v/v, 1 V). The filtrate was concentrated to give 250 g of crude acid (XVII) as a yellow oil, and HPLC and LCMS showed it contained ˜50% of the methyl ester byproduct.

The product was then purified by silica gel column chromatography (5-25% MeOH in DCM) to give 128 g of a mixture of the product acid and the methyl ester byproduct. The mixture (128 g) was dissolved in THF (200 mL) before addition of MeOH (200 mL) and H₂O (200 mL). NaOH (19.5 g, 1.1 eq) was then added, and the mixture was stirred at 25° C. for 12 hours under N₂ atmosphere. HPLC and LCMS showed the methyl ester byproduct was consumed completely. The reaction mixture was concentrated under reduced pressure to remove THF and MeOH to give a residue. The residue was triturated with H₂O (200 mL) at 0° C. for 1 hour and filtered to afford 2-((2-oxo-2-((2-(2-oxopiperidin-1-yl)ethyl)amino)ethyl)thio)acetic acid (XVII) (87 g) as a white solid in 78.6% yield. MS (ESI, pos. ion) m/z: 275.1 (M+1). ¹H-NMR (400 MHz, CDCl₃) δ 7.53 (s, 1H), 3.60 (t, 2H), 3.52 (dd, 2H), 3.42-3.31 (m, 4H), 3.29 (s, 2H), 2.43 (t, 2H), 1.82 (dd, 4H).

### Example 2: Synthesis of GPCR Agonist Peptide (I)

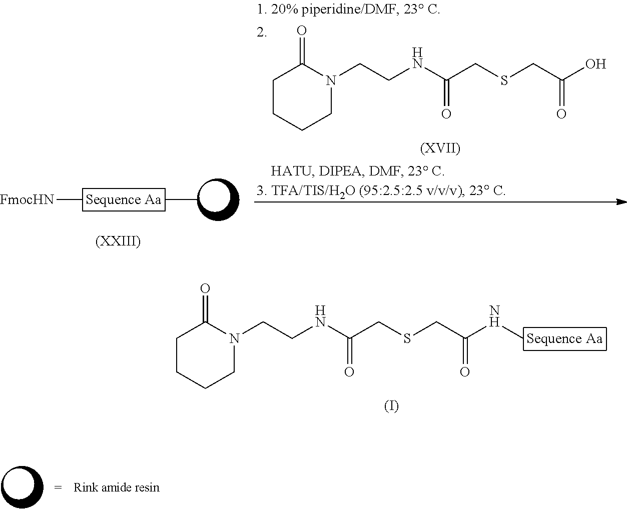

Rink-amide resin-bound amino acid peptide Sequence Aa (XXIII) was prepared according to standard solid-phase peptide synthesis protocols. To peptide-resin conjugate (XXIII) (0.125 mmol/g, 41 mg) in a 6 mL polypropylene tube with an end-cap was added 20% v/v piperidine/DMF (2 mL). The tube was capped and agitated at ambient temperature for 30 minutes, then drained. The resin was washed with DMF (5×3 mL). A solution of carboxylic acid building block (XVII) (36.6 mg, 8.0 eq.) in DMF (2.8 mL) was added to the resin, followed by DIPEA (61 μL, 20 eq.). HATU (80 mg, 12.0 eq.) was then added and the reaction mixture was agitated at ambient temperature for 18 hours. The reaction mixture was drained and the resin washed with DMF (5×3 mL), DCM (5×3 mL), and dried in vacuo for 30 minutes.

The resin was transferred to a 15 mL Falcon tube and 3 mL of cleavage reagent (95:2.5:2.5 v/v/v TFA/TIS/H₂O) was added. The reaction mixture was agitated at ambient temperature for 1 hour. The resin was filtered and washed with TFA (2×3 mL). The combined filtrate and washes were concentrated under reduced pressure to afford a residue, which was triturated with Et₂O (3 mL) to precipitate the peptide. The peptide was re-dissolved in glacial AcOH (2 mL) and purified by preparative HPLC (Phenomenex Jupiter 10 μM Proteo 90 Å LC column, 250×21.2 mm, with flow rate 15 mL/min, gradient of 0-100% acetonitrile in 25 mM aqueous ammonium acetate over 30 minutes) to afford 4.1 mg of (I) as a white solid. ESI-MS (positive ionization) found 1153.3, C₂₁₄H₃₂₇N₄₇O₆₄S (M+4H⁺) requires 1152.8.

### Example 3: Alternative Synthesis of 1-(2-aminoethyl)piperidin-2-one hydrobromide (XXI)

1-(2-aminoethyl)piperidin-2-one hydrobromide (XXI) was synthesized according to the following scheme:

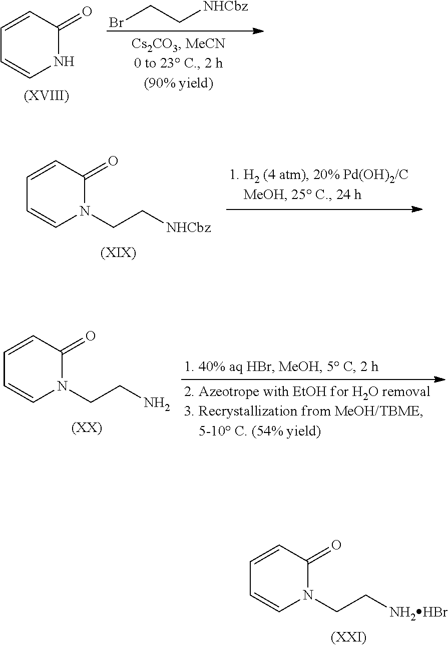

The synthesis commences from commercially available pyridone (XVIII), which is treated under basic conditions with benzyl (2-bromoethyl)carbamate to afford alkylated pyridone (XIX). Catalytic hydrogenation of pyridone (XIX) with concomitant deprotection of the carbobenzyloxy group affords the amine (XX). Amine (XX) is then subjected to hydrobromide salt formation, and recrystallization of this salt affords hydrobromide (XXI).

### Synthesis of Benzyl (2-(2-oxopyridin-1(2H)-yl)ethyl)carbamate (XIX)

To a solution of pyridin-2(1H)-one (CAS: 142-08-5, 5.0 g, 52.1 mmol, 1.0 equiv) in 50 mL MeCN that had been pre-cooled in an ice bath was added benzyl (2-bromoethyl)carbamate (CAS: 53844-02-3, 16.1 g, 62.5 mmol, 1.2 equiv) and Cs₂CO₃ (33.9 g, 104.2 mmol, 2.0 equiv). The reaction mixture was stirred and allowed to warm to 25° C. over two hours. After completion, the reaction mixture was filtered. The filtrate was concentrated to afford a crude product, which was purified by solvating gas chromatography (SGC) (EtOAc/DCM=1:1) to give benzyl (2-(2-oxopyridin-1(2H)-yl)ethyl)carbamate (XIX) (12 g, purity 90%, yield 86%) as a white solid. MS (ESI, pos. ion) m/z: 273.1 (M+1).

Synthesis was also carried out on a larger scale. To a solution of pyridin-2(1H)-one (500 g, 5.2 mol, 1.0 equiv) in 5 L MeCN that had been pre-cooled in an ice bath was added benzyl (2-bromoethyl)carbamate (1620 g, 6.3 mol, 1.2 equiv) and Cs₂CO₃ (2190 g, 6.8 mol, 1.3 equiv). The reaction mixture was stirred at 0-25° C. for 16 hs and filtered. The filtrate was concentrated to afford a crude product, which was purified by SGC (EtOAc/DCM=1:1) to give benzyl (2-(2-oxopyridin-1(2H)-yl)ethyl)carbamate (XIX) (1200 g, purity 90%, yield 86%) as a white solid. MS (ESI, pos. ion) m/z: 273.1 (M+1).

### Synthesis of 1-(2-aminoethyl)piperidin-2-one (XX)

To a solution of benzyl (2-(2-oxopyridin-1(2H)-yl)ethyl)carbamate (XIX) (100 g, 366 mmol, 1.0 equiv) in 500 mL MeOH (HPLC grade) was added 20% Pd(OH)₂/C (20 g). The reaction mixture was stirred at 25° C. for 24 hrs under H₂ (0.4 Mpa). The mixture was filtered and concentrated under reduced pressure to afford crude product 1-(2-aminoethyl)piperidin-2-one (XX) (50 g, purity 80%) as a yellow oil. MS (ESI, pos. ion) m/z: 143.2 (M+1). ¹H NMR (400 MHz, DMSO) δ 3.26 (t, J=5.6 Hz, 3H), 3.21 (d, J=7.0 Hz, 2H), 2.62 (t, J=6.9 Hz, 2H), 2.51-2.49 (m, 2H), 2.18 (t, J=6.1 Hz, 2H), 1.73-1.65 (m, 4H).

The synthesis was also carried out on a larger scale. To a solution of benzyl (2-(2-oxopyridin-1(2H)-yl)ethyl)carbamate (XIX) (200 g, 735 mmol, 1.0 equiv) in 1 L of MeOH (HPLC grade) was added 20% Pd(OH)₂/C (40 g). The mixture was stirred at 25° C. for 24 hrs under H₂ (0.4 Mpa). The mixture was then filtered and concentrated under reduce pressure to afford crude product 1-(2-aminoethyl)piperidin-2-one (XX) (50 g, purity 80%) as a yellow oil. MS (ESI, pos. ion) m/z: 143.2 (M+1).

### Synthesis of 1-(2-aminoethyl)piperidin-2-one hydrobromide (XXI)

To a solution of 1-(2-aminoethyl)piperidin-2-one (XX) (100 g, 704 mmol, 1.0 equiv) in 500 mL of MeOH that had been pre-cooled in an ice bath was added 40% HBr aqueous (169 g, 845 mmol, 1.2 equiv). The reaction mixture was stirred at 5° C. for 2 hours. The solvent was then removed under reduced pressure and the reaction mixture diluted with EtOH (1 L) The mixture was concentrated again to remove water. The resulting crude product was dissolved in MeOH (300 mL) and the solution was cooled in an ice bath. MTBE (600 ml) was added slowly and the reaction mixture was stirred at 5-10° C. for 2 hours, then filtered and the filter cake was washed with MTBE (1 L) and dried to afford product 1-(2-aminoethyl)piperidin-2-one hydrobromide (XXI) (100 g, purity 98%, yield 64%) as a white solid. The chemical structure of the target compound was confirmed by ¹H NMR and LC-MS with the purity >95% (HPLC 214 & 254 nm). MS (ESI, pos. ion) m/z: 143.2 (M+1). ¹H NMR (400 MHz, D₂O) δ 3.61 (t, J=5.9 Hz, 2H), 3.35 (t, J=5.7 Hz, 2H), 3.17 (t, J=5.9 Hz, 2H), 2.33 (t, J=6.3 Hz, 2H), 1.80-1.68 (m, 4H).

The synthesis was also carried out on a larger scale. To a solution of 1-(2-aminoethyl)piperidin-2-one (1000 g, 7.1 mol, 1.0 equiv) in 3 L of MeOH that had been pre-cooled in an ice bath was added 40% HBr aqueous (1690 g, 8.5 mol, 1.2 equiv). The reaction mixture was stirred at 5° C. for 2 hours. The solvent was then removed under reduced pressure and the reaction mixture was diluted with EtOH (3 L). The reaction mixture was concentrated again to remove water. The resulting crude product was dissolved in MeOH (2 L) and the solution was cooled in an ice bath. MTBE (3 L) was then slowly added. The reaction mixture was stirred at 5-10° C. for 2 hours. The reaction mixture was then filtered and the solids were washed with MTBE (2 L) and dried to afford 1-(2-aminoethyl)piperidin-2-one hydrobromide (850 g, purity 99%, yield 54%) as a white solid. MS (ESI, pos. ion) m/z: 143.2 (M+1). ¹H NMR (400 MHz, D₂O) δ 3.56 (t, J=5.9 Hz, 2H), 3.31 (t, J=5.5 Hz, 2H), 3.13 (t, J=5.7 Hz, 2H), 2.29 (t, J=6.2 Hz, 2H), 1.79-1.62 (m, 4H).

### Example 4: Alternative Synthesis of 1-(2-aminoethyl)piperidin-2-one Hydrobromide (XXI)

The synthesis of 1-(2-aminoethyl)piperidin-2-one hydrobromide (XXI) was carried out as shown in the scheme below.

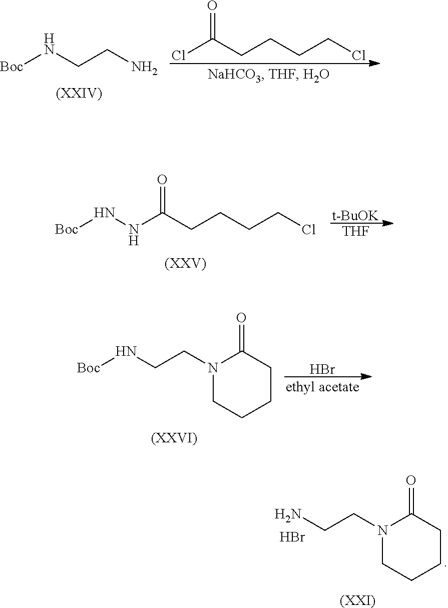

The synthesis commences with the acylation of mono-protected ethylenediamine (XXIV) with an acid chloride to afford the chloropentanoyl amide (XXV), which is treated with base to afford the piperidone (XXVI). Deprotection of the carbamate directly affords the piperidone (XXI), which was purified by recrystallization as in the previous Examples.

### Synthesis of tert-butyl (2-(5-chloropentanamido)ethyl)carbamate

To a solution of N-Boc-ethylenediamine (XXIV) (2.0 kg, 12.5 mol) in THF (10 L) and water (10 L) was added NaHCO₃ (2.1 kg, 25 mol, 2.0 eq). The reaction mixture was cooled to 0° C. 5-Chlorovaleryl chloride (2.3 kg, 15 mol, 1.2 eq.) was added dropwise over two hours. The reaction mixture was stirred at room temperature for one hour at which point the reaction was deemed complete. The reaction mixture was filtered, and the filter cake was washed with water (15 L). The solids were slurried in a solution of EtOAc/petroleum ether (1:50 v/v, 5 L) for one hour then filtered and the filter cake was dried to afford tert-butyl (2-(5-chloropentanamido)ethyl)carbamate (XXV) as a white solid in 86.2% yield (3.0 kg).

This step was performed in two batches, which were combined together for the next step. In the first batch, 2,000 g of starting material was converted into 3,000 g product (86.2% yield). In the second, 4,000 kg of starting material was converted into 6,500 g product (93.4% yield). Mass(m/z): 279.0 [M+H]⁺. ¹H NMR (400 MHz, CDCl₃) δ 6.38 (br, 1H), 5.01 (br, 1H), 3.53-3.56 (t, J=6, 2H), 3.34-3.37 (m, 2H), 3.28 (br, 2H) 2.20-2.24 (t, J=8, 2H), 1.77-1.81 (m, 4H), 1.44 (s, 9H).

### Synthesis of tert-butyl (2-(2-oxopiperidin-1-yl)ethyl)carbamate

To a solution of tert-butyl (2-(5-chloropentanamido)ethyl)carbamate (XXV) (8.5 kg, 30.6 mol) in THF (40 L) was added t-BuOK (6.85 kg, 61.2 mol, 2.0 eq.) while maintaining the temperature at 0° C. The reaction mixture was allowed to warm to ambient temperature and stirred for one hour. The reaction mixture was then quenched with ice water, and extracted by ethyl acetate (2×10 L). The organic phases were combined and washed with brine (20 L) then dried over anhydrous Na₂SO₄ and filtered. The filtrate was concentrated under reduced pressure to afford the crude product which was purified by silica gel chromatography (EtOAc:Petroleum Ether=1:1) to afford 7.16 kg (96.5% yield) of tert-butyl (2-(2-oxopiperidin-1-yl)ethyl)carbamate (XXVI) as a yellow oil.

This step was performed in two batches, which were combined together for the next step. In the first batch, 1,000 g of starting material was converted into 840 g product (96.5% yield). In the second batch, 8,500 g of starting material was converted into 7,160 g of product (96.5% yield). Mass(m/z): 243.1 [M+H]⁺. ¹H NMR (400 MHz, CDCl₃) δ 5.01-5.13 (m, 1H), 3.44-3.47 (m, 2H), 3.26-3.33 (m, 4H), 2.34-2.37 (m, 2H), 1.73-1.79 (m, 4H), 1.40 (s, 9H).

### Synthesis of 1-(2-aminoethyl)piperidin-2-one hydrobromide

To a stirred solution of tert-butyl (2-(2-oxopiperidin-1-yl)ethyl)carbamate (XXVI) (3.0 kg, 12.4 mol) in EtOAc (30 L) was added HBr (7.6 kg, 31 mol, 2.5 eq) at −10° C. under a nitrogen atmosphere. The reaction mixture was allowed to warm to room temperature and stirred for 16 hours, at which point the reaction was deemed complete. The reaction mixture was filtered and then the filter cake was washed with EtOAc (15 L). The filter cake was dissolved in MeOH (10 L) and concentrated under reduced pressure to afford the crude product (4 kg). The crude product was dissolved in MeOH (8 L) and the solution was cooled in an ice bath. MTBE (12 L) was then slowly added into the solution. The reaction mixture was stirred at 5-10° C. for two hours, then filtered and the filter cake was washed with MTBE (5 L) and dried to afford 1-(2-aminoethyl)piperidin-2-one hydrobromide (XXI) as a white solid in 101% yield (2.79 kg).

This step was performed in two batches that were combined together for the next step. In the first batch, 3,000 g of starting material was converted into 2,790 g product (101% yield). In the second batch, 4,820 g starting material was converted into 4,600 g product (104% yield). Mass(m/z): 143.1 [M+H]⁺. ¹H NMR (400 MHz, D₂O) δ 3.54-3.57 (t, J=6, 2H), 3.28-3.31 (t, J=6, 2H), 3.10-3.13 (t, J=6, 2H), 2.26-2.29 (t, J=6, 2H), 1.64-1.74 (m, 4H).

The two batches were combined together and recrystallized with MTBE (15 L) to afford 1-(2-aminoethyl)piperidin-2-one hydrobromide (7.3 kg, purity 99.3%, yield 101.7%) as white solid. Mass(m/z): 143.1 [M+H]⁺. ¹H NMR (400 MHz, D₂O) δ 3.54-3.57 (t, J=6, 2H), 3.29-3.32 (t, J=6, 2H), 3.11-3.14 (t, J=6, 2H), 2.27-2.30 (t, J=6, 2H), 1.67-1.74 (m, 4H). The estimated content of HBr was 46%, indicating that approximately 1.5 mol HBr per mol of product.

### Example 5: Alternative Synthesis of 2-((2-oxo-2-((2-(2-oxopiperidin-1-yl)ethyl)amino)ethyl)thio)acetic acid (XVII)

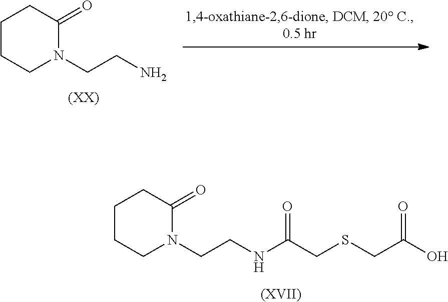

A solution of 1-(2-aminoethyl)piperidin-2-one (4 g, 28.17 mmol) and 1,4-oxathiane-2,6-dione (CAS: 3261-87-8, 4.6 g, 1.2 equivalents) in 40 mL of DCM was stirred at 20° C. for 0.5 hours at which point the reaction was deemed complete. The reaction mixture was concentrated under reduced pressure to afford a pale oil, which was purified by reverse phase column chromatography (MeCN/H₂O, both with 0.5% FA; gradient of 6% to 8%) to afford 2.69 g of the product (XVII) as a white solid (35% yield). MS (ESI, pos. ion) m/z: 275.1 (M+1H+). ¹H NMR (400 MHz, CDCl₃) δ 7.53 (s, 1H), 3.60 (t, 2H), 3.52 (dd, 2H), 3.42-3.31 (m, 4H), 3.29 (s, 2H), 2.43 (t, 2H), 1.82 (dd, 4H).

### Example 6: Alternative Synthesis of 2-((2-oxo-2-((2-(2-oxopiperidin-1-yl)ethyl)amino)ethyl)thio)acetic acid (XVII)

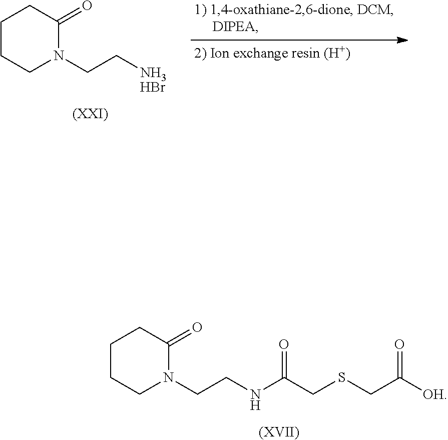

The synthesis was carried out on a molar scale of 0.283 mol. The theoretical yield was 77.6 g (0.283 mol*274.34 g/mol). Cation-exchange resin AG50W-X8 H⁺ form (20-50 mesh) was used. For analysis, a YMC Pro Analytical HPLC (C18 column, 3 μm, 120, 4.6×150 mm) was used with eluent A being 0.1% TFA in H₂O and eluent B being 0.1% TFA in ACN. A gradient of 5-35% B over 20 minutes was used with a flow rate of 1.5 mL/min, an injection volume of 10 μL, and a column temperature of 30° C. Absorbance was measured at 214 nm and 254 nm.

A round bottom flask (3) equipped with a magnetic stir-bar/overhead stirrer was charged with 1-(2-aminoethyl)-2-piperidinone hydrobromide (XXI) (75.8 g, 1.2 eq) and Thiodiglycolic anhydride (37.4 g, 283 mmol). DCM was then added (2830 mL) and the suspension was stirred for 3-7 minutes, after which DIPEA (208 mL, 4.2 eq) was added to the reaction mixture over a time period of 1-3 minutes. The temperature after addition of DIPEA was 25.3° C. The reaction was stirred for at least 30 minutes and up to four hours. Reaction completion was monitored by HPLC at 214 nm and 254 nm.

The product peak area at given time points were as follows: 30 mins: 34.2, 33.1% area; 60 mins: 57.4, 69.9% area; 90 minutes: 56.7, 70.1% area; 2 hours: 56.4, 70.1% area. Based on the peak areas, the reaction was deemed to be complete after 2.5 hours. The reaction mixture was then concentrated under reduced pressure at 20-30° C. until no more condensate was formed and the residue appeared visually dry. Water (2264 mL) was then added and the mixture stirred until a solution was afforded.

Prior to using the ion exchange resin, it was washed with water (2-3 mL USP water per gram of resin). The suspension was stirred for five minutes, then water was drained through a coarse funnel. The wash was repeated five times, each time using fresh USP water. 948 grams of resin were used, with 1900 mL of water used for each wash.

The pre-washed ion exchange resin (H+) was then added in four portions (189.6 g each) to the product solution. After each addition, the slurry was stirred for five minutes then the pH was checked. The pH five minutes after the first addition was 10; after the second, 4; after the third, 1. If the pH was still greater than 2, a 5th ion exchange resin treatment could have been applied. The desired pH was 2 or less.

After final treatment of dissolved product with ion exchange resin, the suspension was filtered through a coarse funnel and the filtrate was collected. The ion exchange resin was then washed with an additional 500 mL of USP water to wash out remaining product.

All sublots were transferred into a glass flask or container. After all product-containing solution has been transferred, the solution was mixed using a stirring rod, then covered with a red plastic cap. The total volume was 2764 mL. The solution was lyophilized using bottle lyophilizers with a condenser temperature from −90.6 to −96 C and vacuum from 50 to 80 mTorr.

Two 2.5 L lyo bottles were used. 64.0 g of product was recovered from first, and 64.6 g was recovered from the second, for a total of 128.6 g. HPLC purity at 214 nm was 84.0% area and at 254 nm was 75.3% area. The product was recovered in 165.6% yield.

### Example 7: Alternative Synthesis of 2-(2-Piperidon-1-yl)-ethylcarbamoylmethylthioacetic Acid

This Example describes a one-step manufacturing process which was successfully applied in the production campaign of 1.7 kg 2-(2-Piperidon-1-yl)-ethylcarbamoylmethylthioacetic acid (XVII). Compared to earlier synthetic schemes, the manufacturing yield increased from 12% to 81% with comparable and even slightly improved product quality.

2-(2-Piperidon-1-yl)-ethylcarbamoylmethylthioacetic acid (XVII) was previously manufactured twice in batch sizes of 324 g and 458 g as described in the following scheme.

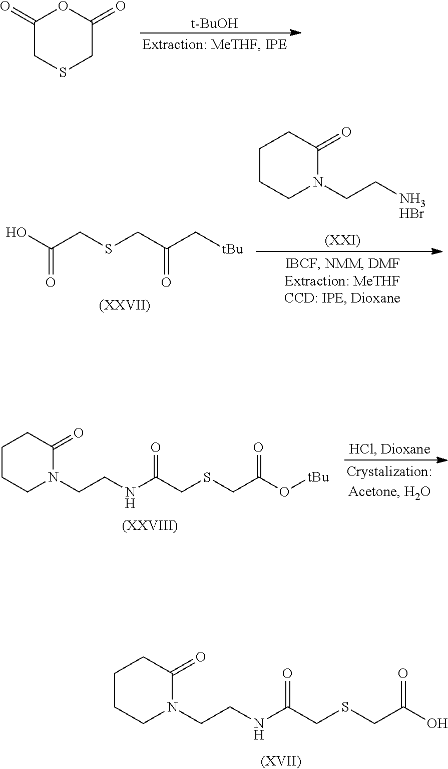

In view of scalability, this process has two major limitations. Firstly, it is a three-step process with a very low overall yield of 12%. Because of selectivity issues and extraction losses, the yield in step 1 is only about 30% which additionally has a negative impact on volume yield of the process. The yield in step 2 is 44%. This also results in a significant loss of the rather expensive reagent 1-(2-aminoethyl)piperidin-2-one hydrobromide on this step.

A second limitation is the purification of the intermediate of step 2 by counter current distribution (CCD). This purification technology limits the maximum batch size to 1-2 kg of material, significantly increases the manufacturing cost, and can result in very long lead times because of limited availability of the specific equipment for CCD.

In view of these issues, the manufacturing process was subjected to development aiming at the establishment of a scalable process. The successful process development resulted in a one-step manufacturing process without use of a CCD purification step shown in the following scheme, which was subsequently applied in the production campaign of 1650 g.

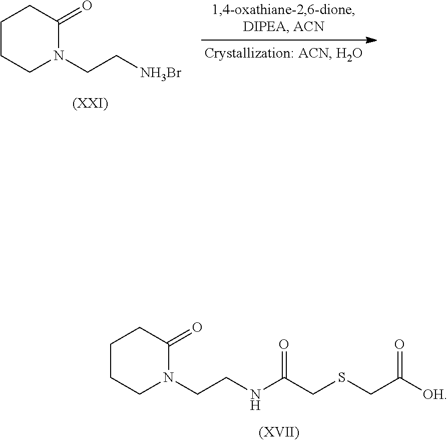

### Use Test for Determination of Stoichiometry

A clean reaction with a minimum of side products is essential for the efficient manufacture of 2-(2-Piperidon-1-yl)-ethylcarbamoylmethylthioacetic acid and isolation of the building block by crystallization. A use test was thus performed with 1-(2-aminoethyl)piperidin-2-one hydrobromide and thiodiglycolic anhydride to correct for inaccuracy in the assay of starting materials. The optimum stoichiometry was found to be 1.07 eq. of 1-(2-aminoethyl)piperidin-2-one hydrobromide and 1.00 eq. of thiodiglycolic anhydride.

### Manufacture of 2-(2-Piperidon-1-yl)-ethylcarbamoylmethylthioacetic acid

In a 90 L glass reactor, 2.00 kg of 1-(2-aminoethyl)piperidin-2-one hydrobromide (8.15 mol, 1.07 eq.) was dissolved under nitrogen atmosphere in 40 L of ACN at 15° C. (external temp. set to 15° C.). Then 1.96 L DIPEA (11.4 mol, 1.50 eq.) was added, maintaining the internal temperature at 15° C. To the resulting white suspension another 10 L of ACN was added and stirring continued for about 30 minutes. In the meantime, a solution of 1.01 kg of thiodiglycolic anhydride (7.62 mol, 1.00 eq.) in 4 L of ACN was prepared in a 10 L flask. The thiodiglycolic anhydride solution was added to the reaction mixture over a period of 30 minutes using a peristaltic pump while maintaining the internal temperature below 20° C. (the external temperature was lowered to min. 5° C. to compensate for the exothermic reaction). The 10 L flask was then rinsed into the reactor via the pump with additional 0.6 L of ACN. The reaction mixture was stirred at an internal temperature of 20-22° C. for 2 hours resulting first in a yellowish solution and then in a white suspension.

The suspension was transferred to a 50 L flask and concentrated under vacuum (80 mbar) with a 50 L rotary evaporator. After evaporation of 44 L ACN, 10.4 kg of a white suspension was obtained and filtered over a vacuum filter. The white solid was washed with ACN (2×4 L) and IPE (2×4 L) and dried under vacuum at 40° C. to yield 1.87 kg of the crude product 2-(2-Piperidon-1-yl)-ethylcarbamoylmethylthioacetic acid.

The crude product was suspended in ACN (20 L) and 1.5 L of deionized water was added. The reaction mixture was heated to 40° C. to afford a yellowish turbid solution, which was subjected to a clarifying filtration. The filter was washed with ACN (2 L), and the combined filtrate concentrated under vacuum at 40° C. After collection of 10 L distillate, 20 L additional ACN was added to the resulting white suspension and distillation continued until 18 L additional distillate was collected. The suspension was then rotated at room temperature for 16 hours and filtered over a vacuum filter to afford a white solid. The white solid was washed with 2×3.8 L of ACN and 2×3.8 L of IPE and dried under vacuum at 40° C. to yield 1698 g (6.19 mol, 81% yield) of the product 2-(2-Piperidon-1-yl)-ethylcarbamoylmethylthioacetic acid as a white solid.

### Summary and Conclusions

The manufacturing process of 2-(2-Piperidon-1-yl)-ethylcarbamoylmethyl-thioacetic acid was subjected to development towards a scalable process suitable for future production in multi kg scale. The process development resulted in a one-step manufacturing process without the need for purification by CCD. The new process was successfully applied in the production campaign of 1.7 kg 2-(2-Piperidon-1-yl)-ethylcarbamoyl-methylthioacetic acid.

### Batch Comparison

A comparison of three manufactured batches of 2-(2-Piperidon-1-yl)-ethylcarbamoyl-methylthioacetic acid is shown in Table 1. The first two batches (1000013161 and 1000032416) were manufactured using the initial three step process, the third batch (1000074879) according to the developed efficient and scalable one step process.

TABLE 1
Comparison of Data for Three Batches of 2-(2-piperidon-
1-yl)-Ethylcarbamoyl-Methylthioacetic Acid

| | Initial process | | Initial Process | | Developed Process | |
|---|---|---|---|---|---|---|
| Batch Size | 324 | g | 458 | g | 1650 | g |
| Yield (corrected for assay) | 12% | | 12% | | 81% | |
| Appearance | White powder | | White powder | | White powder | |
| Monoisotopic Mass (ESI-MS) | 274.1 | u | 274.1 | u | 274.1 | u |

| | | | |
|---|---|---|---|
| HPLC-Purity | 99.1% | 98.6% | 99.2% |
| Main HPLC impurities | | | |
| 2,2′-Thiodiacetic acid | 0.01% | 0.04% | 0.06% |
| Any unspecified impurity | Less than or | Less than or | Less than or |
| | equal to 0.16% | equal to 0.53% | equal to 0.29% |
| Assay (titration) | 78.1% | 80.3% | 99.9% |
| Water content (KF) | 5.9% | 6.3% | 0.1% |

The yield for the new one step process was 81%, an increase of 575% compared to the three-step process (12% yield). The product quality for the one step process was comparable and even slightly better than for the three-step process. The product did not contain a significant amount of water, while in the initial process the material was isolated as a hydrate (ca. 6% water content). The assay of the developed process batch was almost 100%, while previous batches had an assay of only around 80%.

### Example 8: HPLC Purification of 2-((2-(2-oxopiperidin-1-yl)ethylcarbamoyl)methylthio)acetic acid (XVI1)

2-((2-(2-oxopiperidin-1-yl)ethylcarbamoyl)methylthio)acetic acid (XVII) was purified by RP-UPLC on a UPLC equipped with a gradient system, autosampler, and UV detection. The solvent was acetonitrile/water (10/90 (v/v)). The test substance was present in injection at 0.5% (e.g., 50 mg in 10 mL). Eluent A was TFA/acetonitrile/water (0.05/1/99 (v/v/v)). Eluent B was TFA/acetonitrile (0.05/100 (v/v)).

A USP L1, RP C18 Aeris Peptide XB-C18 column (2.6 u; 250×2.1 mm) was used. The gradient program was as shown in Table 2.

TABLE 2
Gradient Program

| Time [min] | Flow [mL/min] | Eluent A [%] | Eluent B[%] |
|---|---|---|---|
| −10.0 | 0.3 | 95 | 5 |
| −1.0 | 0.3 | 95 | 5 |
| −0.9 | 0.3 | 95 | 5 |
| 0.0 | 0.3 | 80 | 20 |
| 10.0 | 0.3 | 60 | 40 |
| 13.0 | 0.3 | 0 | 100 |
| 16.0 | 0.3 | 0 | 100 |

The data acquisition time was 16.0 min, and the run time was 26.0 min. Detection was UV absorbance (λ=240 nm). The column temperature was 25° C., and the autosampler temperature was 5° C. The injection volume was 5 μl.

Impurity 2,2-thiodiacetic acid eluted with a retention time of 3.407 min. The desired product eluted with a retention time of 6.662 min.

1. A method of synthesizing an N-terminal conjugated peptidyl compound of formula (I): 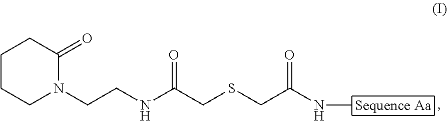 [SMILES: CNC(=O)CSCC(=O)NCCN1CCCCC1=O]
wherein Sequence Aa is a peptide, the method comprising the step of reducing benzyl 2-(2-oxopyridin-1(2H)-yl)ethylcarbamate (XIX): 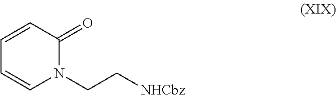 [SMILES: O=C(NCCn1ccccc1=O)OCc1ccccc1]
to 1-(2-aminoethyl)piperidin-2-one (XX): 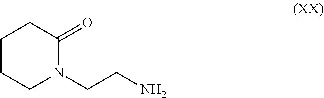
with a catalyst in the presence of H₂.

2. A method of synthesizing a compound of formula (XVI): 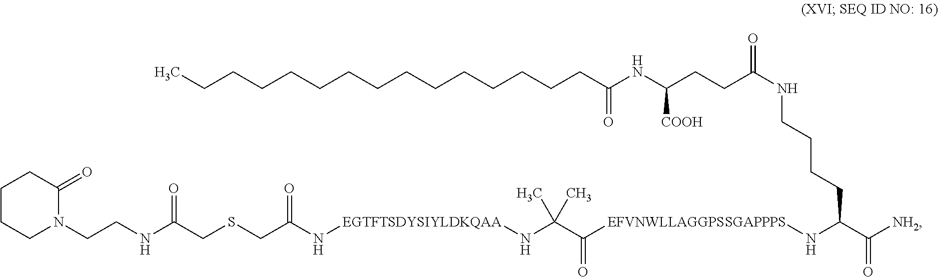
the method comprising the step of reducing benzyl 2-(2-oxopyridin-1(2H)-yl)ethylcarbamate (XIX): 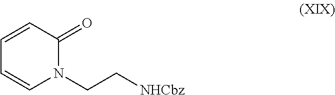 [SMILES: O=C(NCCn1ccccc1=O)OCc1ccccc1]
to 1-(2-aminoethyl)piperidin-2-one (XX): 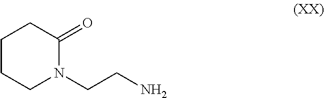
with a catalyst in the presence of H₂.

3. A method of synthesizing 2-((2-oxo-2-((2-(2-oxopiperidin-1-yl)ethyl)amino)ethyl)thio)acetic acid (XVII): 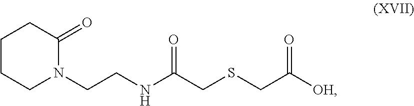 [SMILES: O=C(O)CSCC(=O)NCCN1CCCCC1=O]
the method comprising the step of reducing benzyl 2-(2-oxopyridin-1(2H)-yl)ethylcarbamate (XIX): 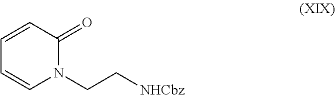 [SMILES: O=C(NCCn1ccccc1=O)OCc1ccccc1]
to 1-(2-aminoethyl)piperidin-2-one (XX): 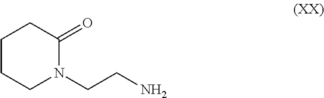
with a catalyst in the presence of H₂.

4. The method of any one of claims 1-3, wherein the catalyst for reduction is palladium on carbon (Pd/C).

5. The method of any one of claims 1-3, wherein the catalyst for reduction is palladium hydroxide on carbon (Pd(OH)₂/C).

6. A method of synthesizing an N-terminal conjugated peptidyl compound of formula (I): 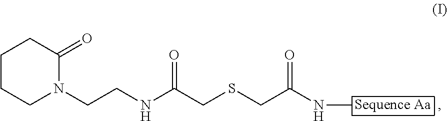 [SMILES: CNC(=O)CSCC(=O)NCCN1CCCCC1=O]
wherein Sequence Aa is a peptide, the method comprising the step of reacting 1-(2-aminoethyl)piperidin-2-one (XX): 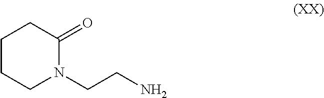
or a salt thereof with 1,4-oxathiane-2,6-dione to produce 2-((2-oxo-2-((2-(2-oxopiperidin-1-yl)ethyl)amino)ethyl)thio)acetic acid (XVII):

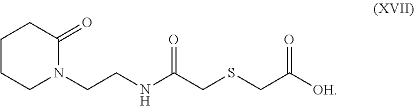 [SMILES: O=C(O)CSCC(=O)NCCN1CCCCC1=O]

7. A method of synthesizing a compound of formula (XVI): 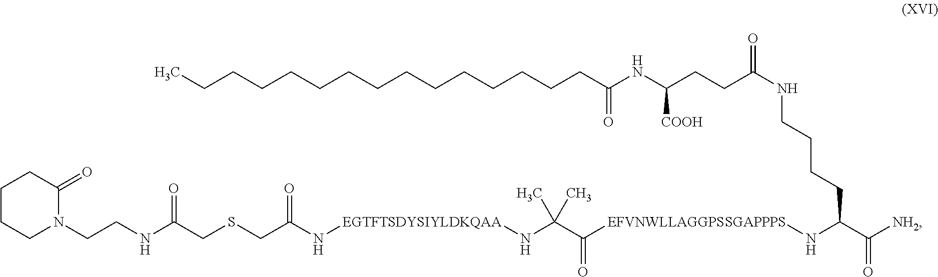
the method comprising the step of reacting 1-(2-aminoethyl)piperidin-2-one (XX): 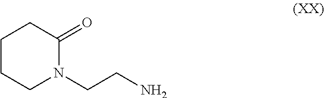
or a salt thereof with 1,4-oxathiane-2,6-dione to produce 2-((2-oxo-2-((2-(2-oxopiperidin-1-yl)ethyl)amino)ethyl)thio)acetic acid (XVII):

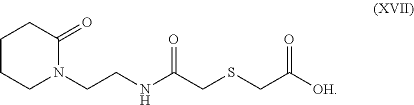 [SMILES: O=C(O)CSCC(=O)NCCN1CCCCC1=O]

8. A method of synthesizing 2-((2-oxo-2-((2-(2-oxopiperidin-1-yl)ethyl)amino)ethyl)thio)acetic acid (XVII): 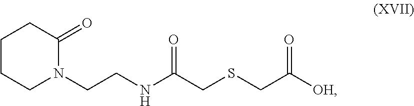 [SMILES: O=C(O)CSCC(=O)NCCN1CCCCC1=O]
the method comprising the step of reacting 1-(2-aminoethyl)piperidin-2-one (XX): 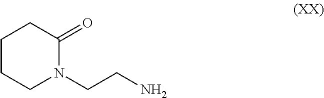
or a salt thereof with 1,4-oxathiane-2,6-dione to produce 2-((2-oxo-2-((2-(2-oxopiperidin-1-yl)ethyl)amino)ethyl)thio)acetic acid (XVII):

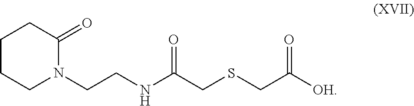 [SMILES: O=C(O)CSCC(=O)NCCN1CCCCC1=O]

9. The method of any one of claims 6-8, wherein the hydrobromide salt of 1-(2-aminoethyl)piperidin-2-one (XX) is reacted with 1,4-oxathiane-2,6-dione.

10. The method of claim 1 or 6, wherein Sequence Aa comprises the formula W-R⁵, wherein W is a peptide sequence and R^{s} is conjugated to the C-terminus of W, wherein W comprises the following sequence:
(SEQ ID NO: 1)
EGT(Xaa4)(Xaa5)SD(Xaa8)S(Xaa10)(Xaa11)(Xaa12)
(Xaa13)(Xaa14)(Xaa15)(Xaa16)(Xaa17)(Xaa18)
(Xaa19)(Xaa20)(Xaa21)(Xaa22)WL(Xaa25)(Xaa26)
(Xaa27)GPSSGAPPP(Xaa37);
wherein:
Xaa4 is F;
Xaa5 is T or I;
Xaa8 is Y, V, L, or K*;
Xaa10 is I or S;
Xaa11 is Y, Y*, Q, A, or (Aib);
Xaa12 is L, M, or L*;
Xaa13 is D or E;
Xaa14 is K, G, R, or E;
Xaa15 is Q or I;
Xaa16 is A, H, or R;
Xaa17 is A, Q, or V;
Xaa18 is A, (Aib), K*, K, or Q;
Xaa19 is A, D, E, (Aib), or L;
Xaa20 is F or A;
Xaa21 is V or I;
Xaa22 is N, A, Q, K*, or E;
Xaa25 is I, L or V;
Xaa26 is A, K, or I;
Xaa27 is Q-R, G-R-G-K*, Q, or G; and
Xaa37 is S or absent; and
R^{s} is a C-terminal amino acid amide or a C-terminal amino acid that is optionally substituted with 1 or 2 modifying groups selected from an acyl group and a PEG group.

11. The method of claim 10, wherein W comprises the following sequence:
(SEQ ID NO: 4)
EGTFTSDYSIYLDKQAA(Aib)EFVNWLLAGGPSSGAPPPS.

12. The method of claim 10 or 11, wherein R⁵ is a C-terminal lysyl amide residue that is optionally substituted with 1 or 2 modifying groups selected from an acyl group and a PEG group.

13. The method of any one of claims 10-12, wherein R⁵ comprises formula (II): 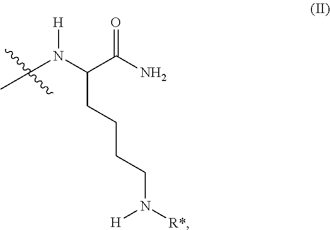
and wherein R* comprises the structure (IV):

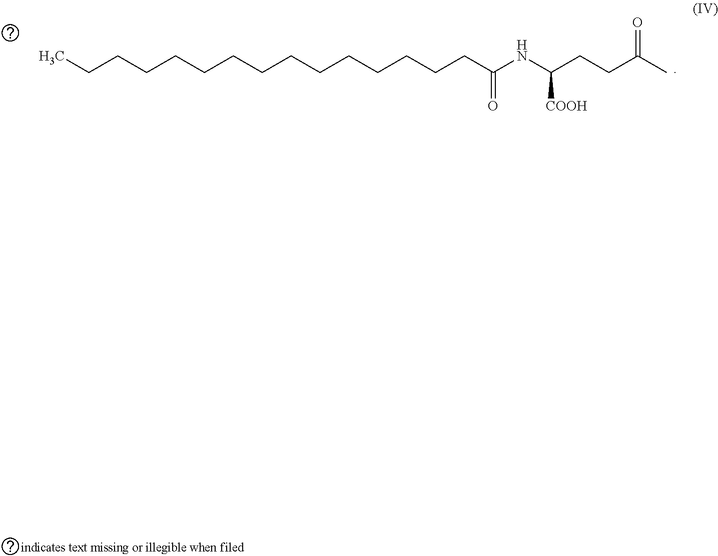 [SMILES: CCCCCCCCCCCCCCCC(=O)N[C@@H](CCC(C)=O)C(=O)O]

14. The method of any one of claims 10-13, wherein W-R⁵ comprises the structure (V):

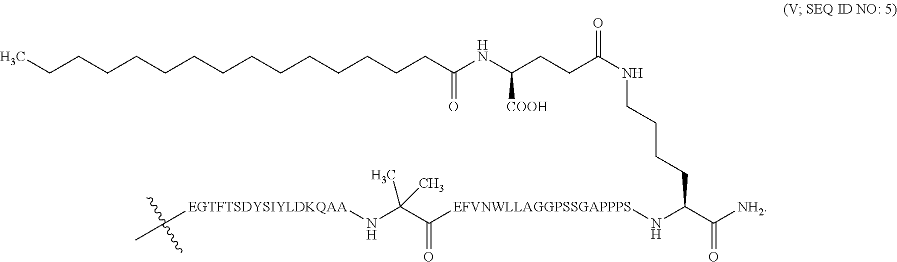

15. The method of any one of claims 1, 6, and 10-14, further comprising the steps of
(i) treating the resin-bound peptide of formula (XXIII): 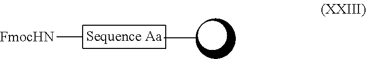
with 20% piperidine in DMF, and
(ii) reacting the product of step (i) with 2-((2-oxo-2-((2-(2-oxopiperidin-1-yl)ethyl)amino)ethyl)thio)acetic acid (XVII): 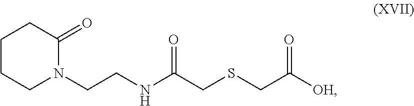 [SMILES: O=C(O)CSCC(=O)NCCN1CCCCC1=O]
under amide bond-forming conditions.

16. The method of any one of claims 2, 7, and 14, further comprising the steps of
(i) treating the resin-bound peptide of formula (XXII): 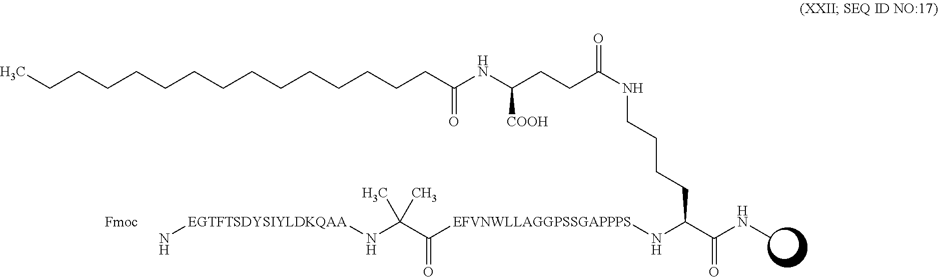
with 20% piperidine in DMF, and
(ii) reacting the product of step (i) with 2-((2-oxo-2-((2-(2-oxopiperidin-1-yl)ethyl)amino)ethyl)thio)acetic acid (XVII): 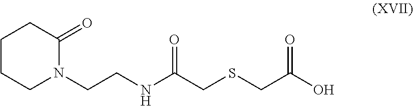 [SMILES: O=C(O)CSCC(=O)NCCN1CCCCC1=O]
under amide bond-forming conditions.

17. The method of claim 15 or 16, wherein the amide bond-forming conditions comprise use of a coupling reagent.

18. The method of claim 17, wherein the coupling reagent is hexafluorophosphate azabenzotriazole tetramethyl uronium (HATU).

19. The method of any one of claims 1-18, wherein the method further comprises the step of reacting pyridin-2(1H)-one (XVIII): 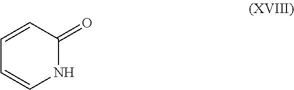
with benzyl (2-bromoethyl)carbamate in the presence of a non-nucleophilic base to produce benzyl 2-(2-oxopyridin-1(2H)-yl)ethylcarbamate (XIX):

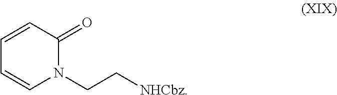 [SMILES: O=C(NCCn1ccccc1=O)OCc1ccccc1]

20. The method of claim 19, wherein the non-nucleophilic base is sodium hydride.

21. The method of any one of claims 1-20, wherein the method further comprises the step of reacting 1-(2-aminoethyl)piperidin-2-one (XX): 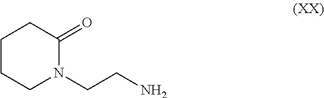
with hydrobromic acid to produce 1-(2-aminoethyl)piperidin-2-one hydrobromide (XXI):

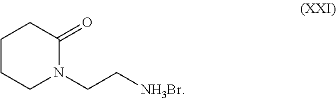

22. The method of any one of claims 1-5 and 11-21, wherein the method further comprises the step of reacting 1-(2-aminoethyl)piperidin-2-one hydrobromide (XXI): 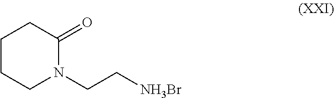
with 1,4-oxathiane-2,6-dione to produce 2-((2-(2-oxopiperidin-1-yl)ethylcarbamoyl)methylthio)acetic acid (XVII):

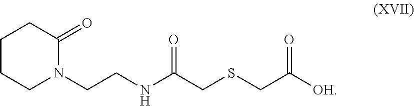 [SMILES: O=C(O)CSCC(=O)NCCN1CCCCC1=O]

23. A compound which is benzyl 2-(2-oxopyridin-1(2H)-yl)ethylcarbamate (XIX): 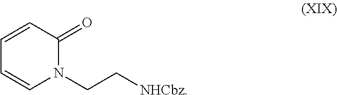 [SMILES: O=C(NCCn1ccccc1=O)OCc1ccccc1]
